(19) United States
(12) Patent Application Publication
(10) Pub. No.: US 2025/0025080 A1
(43) Pub. Date: Jan. 23, 2025

(54) ALWAYS UNIFIED SYSTEM FOR TESTING ANALYTE LEVELS, MODULES AND METHODS THEREOF

(71) Applicant: SMARTEST TECHNOLOGIES LTD., Tel Aviv (IL)

(72) Inventors: David WEINTRAUB, Ramat Gan (IL); Ariel HOROWITZ, Qiryat Ono (IL); Eliezer ANAVI, Herzliya (IL); Amit BARAK, Tel Aviv (IL); Alexey RUBINCHIK, Rishon le Zion (IL)

(21) Appl. No.: 18/904,128

(22) Filed: Oct. 2, 2024

Related U.S. Application Data
(63) Continuation-in-part of application No. PCT/IL2023/050355, filed on Apr. 3, 2023.
(60) Provisional application No. 63/326,875, filed on Apr. 3, 2022.

(51) Int. Cl. A61B 5/157; A61B 5/00; A61B 5/1477; A61B 5/15

(52) U.S. Cl. CPC A61B 5/157; A61B 5/1477; A61B 5/150022; A61B 5/150068; A61B 5/150114; A61B 5/150175; A61B 5/150412; A61B 5/742; A61B 2560/0214; A61B 2560/045; A61B 2560/0462

(57) ABSTRACT

The present invention discloses means and methods for reducing or eliminating pain during testing analyte levels of bodily fluids, including glucose, comprising a chip configured for both (i) to process electrical pulses obtained from a communicable analyzing strip, and (ii) to communicate the processed digital data. Pain reduction mechanisms accommodated by the device may include a finger clamping mechanism and/or an ergonomically contoured elastic finger-pad. The invention also discloses means and methods for body piercing, by an integrated diagnostic device comprising a lancet reversibly movable in parallel with a longitudinal axis from a first retract position, to a fully extended third position within the dermis and above and outside the venuole plexus, via a second position, located within the stratum cornea, and then back, to a final retracted fourth position; wherein the velocity of said lancet in its way from said first to said second position is relatively slow.

### CROSS-REFERENCE TO RELATED PUBLICATIONS

This application is a Continuation-in-Part of International PCT Patent Application No. PCT/IL2023/050355 having International filing date of Apr. 3, 2023, which claims the benefit of priority of U.S. Provisional Patent Application No. 63/326,875, filed Apr. 3, 2022. All of these documents are incorporated by reference in their entirety.

### FIELD OF THE INVENTION

The present invention pertains to an always unified real-time system for reducing and preventing pain during testing analyte levels in a fluid sample, e.g., glucose levels in blood, modules, devices and methods thereof.

### BACKGROUND OF THE INVENTION

The quantitative determination of analytes in body fluids is of great importance in the diagnoses and maintenance of certain physiological abnormalities. For example, lactate, cholesterol, and bilirubin should be monitored in certain individuals. In particular, determining glucose in body fluids is important to diabetic individuals who must frequently check the glucose level in their body fluids to regulate the glucose intake in their diets. This is because diabetes can lead to serious complications if glucose levels are not well-regulated, including cardiovascular diseases, nerve damage, and kidney issues. Generally, each test involves cartridges that contain a number of test sensors used to allow users to carry multiple strips around within a single-housing object. The multiple stages required to perform such tests can be long and difficult. In some cases, once the testing package is opened, the user needs to be sure that the sensor is not damaged or contaminated as it is being placed into a sensor holder and used to test the blood sample. Further, once the sensor is placed in the sensor holder, a fluid sample must be collected and applied to the sensor More than 34M American have diabetes (2022), and more than 88M have prediabetes. Day by day, some 4,110 new people in the US are diagnosed with diabetes. Diabetes is characterized by short, medium-and long-term relevant diseases, including amputation (some 256 new cases per day) and end-stage kidney diseases (159 new cases a day). hence, annual costs of diagnosed diabetes in the US is huge, namely about $327B.

Fear of injections is estimated to affect a majority of younger children; the percentage of those fearing injections decreases to 20%-50% in adolescents and 20%-40% in young adults, sec McLenon, J., & Rogers, M. A. (2019). The fear of needles: A systematic review and meta-analysis. Journal of advanced nursing, 75 (1), 30-42. Some 32% of caregivers reported fear of injections in their child diagnosed with diabetes aged 6-17 years old, see Cemeroglu, A. P., et al. “Comparison of the Expectation of Caregivers and Children with Type 1 Diabetes Mellitus Doe Independence in Diabetes Care-Related Tasks.” Endocrine Practice 20.7 (2014): 629-637. Fear is not always significantly lower in adults: Needle fear exists on a continuum of severity from dislike and discomfort to phobia. In the general adult population, the prevalence of injection fear was found to be 16.1% in the Netherlands. The frequency of needle phobia in general adult populations is not less common. Experts estimate that needle-fear occurred in 16% of adult patients, 27% of hospital employees, 18% of workers at long-term care facilities and 8% of healthcare workers at hospitals, see McLenon, J., and M AM Rogers. “The fear of needles: A systematic review and meta-analysis.” Journal of advanced nursing 75.1 (2019): 30-42.

Commercially available glucometers are provided useful for indicating the disease, yet not for avoiding the condition while it is only a metabolic deviation. It is a hence a long felt need to have a small and portable, ready-to-use, inexpensive, without the burden to “keep and carry” it, incorporating pain reduction or prevention apparatus, suitable as a single-use, disposable system, for testing the level of glucose in the blood, characterized by both: (i) non-tedious and intuitively-operated, free of pain and anxiety for pain or discomfort, and (ii) improved user experience hence compliance for the test. This new type of glucometer encourages most of the population, the so-called ‘healthy people’, namely those who have had none previous indications for the disease, to test their glucose levels periodically, and hence enable early detection of glucose metabolism deviation and avoidance of a diabetic state.

### SUMMARY OF THE INVENTION

Generally, the invention concerns devices and methods thereof for reducing and preventing pain during body fluid sampling, such as blood collection. By incorporating pain reduction/prevention apparatus, such as clamping mechanisms or ergonomically contoured elastic rigid finger-pads, these devices aim to enhance patient comfort and minimize discomfort. The methods leverage careful site selection and real-time monitoring to optimize the sampling process, ensuring both efficiency and a gentler experience. This approach not only alleviates anxiety associated with fluid sampling but also promotes better compliance and cooperation from patients, ultimately improving overall healthcare experiences.

In one aspect of the invention, a unified real-time system for analyte levels in a fluid sample, including blood glucose level. The invention concerns a novel blood-test system comprising a housing having a lancet and a finger-clamping mechanism. The lancet is provided in adjacent location to an analyzing strip. This all-in-one blood glucose testing system is characterized by a chip configured to process electrical signals obtained from the analyzing strip, and further to communicate the processed digital data.

In another aspect of the invention, the system as defined above, wherein the unified all-in-one system for testing glucose level in the blood is disposable, miniature, easy to use, no need for “keep and carry” burden, both non-tedious and intuitively-operated, free of pain and anxiety for pain or discomfort, characterized by improved user experience, being inexpensive for both the insurer (such as HMO, etc.) and the user. As accurate tests results are immediately transferred online to both the user and to any predefined remote location, e.g., HMO and professionals thereof, the endless chain of user-point of care-laboratory-HMO-user is saved, so that efforts, costs, errors and time consumption are significantly reduced.

It is thus an object of the invention to disclose a system, device, module and a kit (collectively termed as “system”) for testing analyte levels, including glucose, comprising a chip configured for both (i) to process electrical pulses obtained from a communicable analyzing strip, and (ii) to communicate the processed digital data.

Another object of the invention is to discloses the system as defined above, wherein at least one of the following is held true: the chip is an application-specific integrated circuit (ASIC); the system further comprising a lancet; the system further comprising a lancet and one or more analyte/analytes analyzing strips; the system further comprising a housing accommodating the chip, a lancet and one or more analyte or analytes analyzing strips; the system further comprising a housing accommodating the chip, a lancet and one or more analyte or analytes analyzing strips; and one or more members of a group consisting of power source, power-source adapter, power source input means; means for input communication, means for output communication and any combination thereof; the system is an always unified apparatus; the system is an all-in-one, always unified apparatus for sugar level testing is disposable; the system further comprising an ergonomically contoured elastic finger-pad is located adjacent to the lancet's sharp end; the system further comprising a finger-pad is a concaved member, having an outer rigid (elastic) protruding circumference, mounted on an elastic base, shaped by means of size and shape to snugly fit the finger pad, and an inner hollow recess configured to accept the sharp end of the lancet; the system further comprising an ergonomically contoured clastic rigid finger-pad; the system further is characterized by a main longitudinal axis A:A, comprising a rigid (an elastic) finger-pad, mounted on an elastic base, configured to deform, narrow or otherwise retract along the axis; the system further is characterized by a main longitudinal axis A:A, comprising a lancet reversibly movable in parallel with the axis from a first retract position, to a fully extended third position within the dermis and above and outside the venuole plexus, via a second position, located within the stratum cornea, and then back, to a final retracted (safely locked-) forth position; wherein the velocity of the lancet in its way from the first to the second position (V_{1st-2nd}) is relatively slow, namely V_{1st-2nd}≥2 m/s; the system further comprising a finger-pad having an outer movable protruding circumference shaped by means of size and shape to snugly fit the finger pad, and an inner hollow recess; the system further comprising a finger-pad configured by means of its size, shape, elasticity and materials, to ensure either or both, (i) that body portion to be pierced is provided in a tension so that it is held firmly and the skin is thin and less elastic as possible, and (ii) the tension in effective to increase blood pressure in at least 5 mmHg as compared to local blood pressure before tensioning the body portion; the system further comprising a finger-pad wherein elasticity of the finger-pad is ranging from Young's modulus: 10³ to 5*10⁹Pa; from 0 to 45 Shore A; or 0 to 80 Shore 00; the system further comprising an elastic finger-pad wherein the finger-pad is configured to deformed when pressed by a body portion to be pierced to a width W_{FPR}, equal or smaller than a function of the width of the epidermis W_{EPI}, so that W_{FPR}≤ϕ*W_{EPI}, 0.25≤ϕ≥0.75; the system further comprising an clastic finger-pad wherein the finger-pad is configured to deformed when pressed by a body portion to be pierced to a width W_{FPR}, equal or smaller than a function of the width of the stratum corneum W_{SC}, so that c≤φ*W_{SC}, 0.3≤℠≥0.6; the system further comprising an elastic finger-pad wherein the finger pad is configured to one or more members of a group consisting of (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin; and the system further comprising a lancet reversibly movable from a first retract position, to a fully extended third position within the dermis and above and outside the venuole plexus, via a second position, located within the stratum cornea, and then back, to a final retracted (safely locked-) forth position; wherein the velocity of the lancet in its way from the first to the second position (V_{1st-2nd}) is relatively slow, namely V_{1st-2nd}≥2 m/s.

Another object of the invention is to disclose a method for testing analyte levels, including glucose levels. The method comprises steps of (i) providing a unified system within a housing having a lancet; (ii) providing the lancet with a fluid connection with an analyzing strip; (iii) configuring a chip to process electrical pulses obtained from the strip, and then (iv) by means of the chip, communicating the processed digital data, by wire or wirelessly, with a cell phone or computer. In this method, at least one of the following is held true: the method further comprising step of providing the chip as an application-specific integrated circuit (ASIC); the method further comprising step of providing the system with a lancet; the method further comprising step of providing a lancet and one or more analyte/analytes analyzing strips; the method further comprising step of providing a housing accommodating the chip, a lancet and one or more analyte or analytes analyzing strips; the method further comprising step of providing a housing accommodating the chip, a lancet and one or more analyte or analytes analyzing strips; and one or more members of a group consisting of power source, power-source adapter, power source input means; means for input communication, means for output communication and any combination thereof; the method further comprising step of providing the system as an always unified apparatus; the method further comprising step of providing system as an all-in-one, always unified apparatus for sugar level testing is disposable; the method further comprising step of providing the same as an ergonomically contoured elastic finger-pad is located adjacent to the lancet's sharp end; the method further comprising step of providing a finger-pad is a concaved member, having an outer elastic protruding circumference shaped by means of size and shape to snugly fit the finger pad, and an inner hollow recess configured to accept the sharp end of the lancet; the method further comprising step of providing an ergonomically contoured elastic finger-pad; the method further comprising step of providing a main longitudinal axis A:A, comprising an elastic finger-pad, configured to deform, narrow or otherwise retract along the axis; the method further comprising step of providing a main longitudinal axis A:A, comprising a lancet reversibly movable in parallel with the axis from a first retract position, to a fully extended third position within the dermis and above and outside the venuole plexus, via a second position, located within the stratum cornea, and then back, to a final retracted (safely locked-) forth position; wherein the velocity of the lancet in its way from the first to the second position (V_{1st-2nd}) is relatively slow, namely V_{1st-2nd}≥2 m/s; the method further comprising step of providing a finger-pad having an outer movable protruding circumference shaped by means of size and shape to snugly fit the finger pad, and an inner hollow recess; the method further comprising step of providing a finger-pad configured by means of its size, shape, elasticity and materials, to ensure either or both, (i) that body portion to be pierced is provided in a tension so that it is held firmly and the skin is thin as possible, and (ii) the tension in effective to increase blood pressure in at least 5 mmHg as compared to local blood pressure before tensioning the body portion; the method further comprising step of providing a finger-pad wherein elasticity of the finger-pad is ranging from Young's modulus: 10³ to 5*10⁹Pa; from 0 to 45 Shore A; or 0 to 80 Shore 00; the method further comprising step of providing an elastic finger-pad wherein the finger-pad is configured to deformed when pressed by a body portion to be pierced to a width W_{FPR}, equal or smaller than a function of the width of the epidermis W_{EPI}, so that W_{FPR}≤ϕ*W_{EPI}, 0.25≤ϕ≥0.75; the method further comprising step of providing an elastic finger-pad wherein the finger-pad is configured to deformed when pressed by a body portion to be pierced to a width W_{FPR}, equal or smaller than a function of the width of the stratum corneum W_{SC}, so that c≤φ*W_{SC}, 0.3≤φ≥0.6; the method further comprising step of providing an elastic finger-pad wherein the finger pad is configured to one or more members of a group consisting of (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin; and the method further comprising step of providing a lancet reversibly movable from a first retract position, to a fully extended third position within the dermis and above and outside the venuole plexus, via a second position, located within the stratum cornea, and then back, to a final retracted (safely locked-) forth position; wherein the velocity of the lancet in its way from the first to the second position (V_{1st-2nd}) is relatively slow, namely V_{1st-2nd}≥2 m/s.

Another object of the invention is to disclose the use of a unified system method for testing analyte levels, including glucose levels. The use comprises steps of (i) providing the all-in-one system within a housing having a lancet; (ii) providing the lancet with a fluid connection with an analyzing strip; (iii) configuring a chip to process electrical pulses obtained from the strip, and then (iv) by means of the chip, communicating the processed digital data.

It is in the scope of the invention wherein at least one of the following is held true: (a) the use of the system as defined in any of the above comprises step of providing the chip as an application-specific integrated circuit (ASIC); the use of the system as defined in any of the above comprises step of providing the system with a lancet; the use of the system as defined in any of the above comprises step of providing a lancet and one or more analyte/analytes analyzing strips; the use of the system as defined in any of the above comprises step of providing a housing accommodating the chip, a lancet and one or more analyte or analytes analyzing strips; the use of the system as defined in any of the above comprises step of providing a housing accommodating the chip, a lancet and one or more analyte or analytes analyzing strips; and one or more members of a group consisting of power source, power-source adapter, power source input means; means for input communication, means for output communication and any combination thereof; the use of the system as defined in any of the above comprises step of providing the system as an always unified apparatus; the use of the system as defined in any of the above comprises step of providing system as an all-in-one, always unified apparatus for sugar level testing is disposable; the use of the system as defined in any of the above comprises step of providing the same as an ergonomically contoured elastic finger-pad is located adjacent to the lancet's sharp end; the use of the system as defined in any of the above comprises step of providing a finger-pad is a concaved member, having an outer elastic protruding circumference shaped by means of size and shape to snugly fit the finger pad, and an inner hollow recess configured to accept the sharp end of the lancet; the use of the system as defined in any of the above comprises step of providing an ergonomically contoured clastic finger-pad; the use of the system as defined in any of the above comprises step of providing a main longitudinal axis A:A, comprising an elastic finger-pad, configured to deform, narrow or otherwise retract along the axis; the use of the system as defined in any of the above comprises step of providing a main longitudinal axis A:A, comprising a lancet reversibly movable in parallel with the axis from a first retract position, to a fully extended third position within the dermis and above and outside the venuole plexus, via a second position, located within the stratum cornea, and then back, to a final retracted (safely locked-) forth position, the velocity of the lancet in its way from the first to the second position (V_{1st-2nd}) is relatively slow, namely V_{1st-2nd}≥2 m/s; the use of the system as defined in any of the above comprises step of providing a finger-pad having an outer movable protruding circumference shaped by means of size and shape to snugly fit the finger pad, and an inner hollow recess; the use of the system as defined in any of the above comprises step of providing a finger-pad configured by means of its size, shape, elasticity and materials, to ensure either or both, (i) that body portion to be pierced is provided in a tension so that it is held firmly and the skin is thin as possible, and (ii) the tension in effective to increase blood pressure in at least 5 mmHg as compared to local blood pressure before tensioning the body portion; the use of the system as defined in any of the above comprises step of providing a finger-pad wherein elasticity of the finger-pad is ranging from Young's modulus: 10³ to 5*10⁹Pa; from 0 to 45 Shore A; or 0 to 80 Shore 00; the method further comprising step of providing an elastic finger-pad wherein the finger-pad is configured to deformed when pressed by a body portion to be pierced to a width W_{FPR}, equal or smaller than a function of the width of the epidermis W_{EPI}, so that W_{FPR}≤ϕ*W_{EPI}, 0.25≤ϕ≅0.75; the use of the system as defined in any of the above comprises step of providing an elastic finger-pad wherein the finger-pad is configured to deformed when pressed by a body portion to be pierced to a width W_{FPR}, equal or smaller than a function of the width of the stratum corneum W_{SC}, so that c≤φ*W_{SC}, 0.3≤φ≥0.6; the use of the system as defined in any of the above comprises step of providing an elastic finger-pad wherein the finger pad is configured to one or more members of a group consisting of (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin; and the use of the system as defined in any of the above comprises step of providing a lancet reversibly movable from a first retract position, to a fully extended third position within the dermis and above and outside the venuole plexus, via a second position, located within the stratum cornea, and then back, to a final retracted (safely locked-) forth position; wherein the velocity of the lancet in its way from the first to the second position (V_{1st-2nd}) is relatively slow, namely V_{1st-2nd}≥2 m/s.

It is also an object of the invention to disclose a body piercing device, characterized by an ergonomically contoured elastic finger-pad. Another object of the invention is to disclose the medical device as defined above, having a main longitudinal axis A:A, elastic finger-pad, configured to deform, narrow or otherwise retract along the axis. Another object of the invention is to disclose the device as defined in any of the above, wherein the finger-pad is a concaved member, having an outer elastic protruding circumference shaped by means of size and shape to snugly fit the finger pad, and an inner hollow recess. Another object of the invention is to disclose the device as defined in any of the above, wherein the elasticity of the finger-pad is ranging from Young's modulus: about 10³ to about 5*10⁹ Pa; from 0 to about 45 Shore A; or 0 to about 80 Shore 00. Another object of the invention is to disclose the device as defined in any of the above, wherein the finger-pad is having an outer movable protruding circumference shaped by means of size and shape to snugly fit the finger pad, and an inner hollow recess. Another object of the invention is to disclose the device as defined in any of the above, wherein finger-pad comprises a static portion and an outer dynamically active protruding circumference, interconnected by means selected from a group consisting a spring, sliding mechanism, rack and pinion mechanism, screw mechanism. pneumatic mechanism, motor, and any combination thereof. Another object of the invention is to disclose the device as defined in any of the above, wherein the elastic finger-pad is configured to deformed along the axis to a width W_{FPR}, equal or smaller than a function of the width of the epidermis W_{EPI}, so that W_{FPR}≤ϕ*W_{EPI}, about 0.25≤ϕ≥ about 0.75. Another object of the invention is to disclose the device as defined in any of the above, wherein the elastic finger-pad is configured to deformed along the axis to a width W_{FPR}, equal or smaller than a function of the width of the stratum corneum W_{SC}, so that W_{FPR}≤φ*W_{SC}, 0.3≤φ≥ about 0.6. Another object of the invention is to disclose the device as defined in any of the above, wherein the elastic finger-pad is having an aperture in which a lancet is movable along the main longitudinal axis A:A Another object of the invention is to disclose a body piercing device, having a main longitudinal axis A:A; comprising a lancet reversibly movable in parallel with the axis from a first retract position, to a fully extended third position within the dermis and above and outside the venuole plexus, via a second position, located within the stratum cornea, and then back, to a final retracted (safely locked-) forth position; wherein the velocity of the lancet in its way from the first to the second position (V_{1st-2nd}) is relatively slow, namely V_{1st-2nd}≥ about 2 m/s. Another object of the invention is to disclose such a device, wherein the acceleration of the lancet in its way from the first to the second position (A_{1st-2nd}) is relatively low, namely A_{1st-2nd}≥ about 4 m/s². Another object of the invention is to disclose the device as defined in any of the above, wherein the device further characterized by an ergonomically contoured elastic finger-pad. Another object of the invention is to disclose the device as defined in any of the above, wherein the device is having a main longitudinal axis A:A, and the elastic finger-pad, configured to deform, narrow or otherwise retract along the axis. Another object of the invention is to disclose the device as defined in any of the above, wherein the finger-pad is a concaved member, having an outer elastic protruding circumference shaped by means of size and shape to snugly fit the finger pad, and an inner hollow recess. Another object of the invention is to disclose the device as defined in any of the above, wherein the elasticity of the finger-pad is ranging from Young's modulus: about 10³ to about 5*10⁹ Pa; from 0 to about 45 Shore A; or 0 to about 80 Shore 00. Another object of the invention is to disclose the device as defined in any of the above, wherein the finger-pad is having an outer movable protruding circumference shaped by means of size and shape to snugly fit the finger pad, and an inner hollow recess. Another object of the invention is to disclose the device as defined in any of the above, wherein finger-pad comprises a static portion and an outer dynamically active protruding circumference, interconnected by means selected from a group consisting a spring, sliding mechanism, rack and pinion mechanism, screw mechanism. pneumatic mechanism, motor, and any combination thereof. Another object of the invention is to disclose the device as defined in any of the above, wherein the elastic finger-pad is configured to deformed along the axis to a width W_{FPR}, equal or smaller than a function of the width of the epidermis W_{EPI}, so that W_{FPR}≤ϕ* W_{EPI}, about 0.25≤ϕ≥ about 0.75. Another object of the invention is to disclose the device as defined in any of the above, wherein the clastic finger-pad is configured to deformed along the axis to a width W_{FPR}, equal or smaller than a function of the width of the stratum corneum W_{SC}, so that W_{SC}φ*W_{SC}, about 0.3≤ϕ≥ about 0.6.

Another object of the invention is to disclose a method for piercing the body. The method comprises step of providing a body piercing device with an ergonomically contoured elastic finger-pad. Another object of the invention is to disclose such a method wherein the method further comprising step of configuring the elastic finger-pad to deform, narrow or otherwise retract along the device's main longitudinal axis A:A. Another object of the invention is to disclose the method as defined in any of the above, wherein the method further comprises step of configuring the finger-pad as a concaved member, having an outer elastic protruding circumference shaped by means of size and shape to snugly fit the finger pad, and an inner hollow recess. Another object of the invention is to disclose the method as defined in any of the above, wherein the elasticity of the finger-pad is ranging from Young's modulus: about 10³ to about 5*10⁹ Pa; from 0 to about 45Shore A; or 0 to about 80 Shore 00. Another object of the invention is to disclose the method as defined in any of the above, wherein the finger-pad is having an outer movable protruding circumference shaped by means of size and shape to snugly fit the finger pad, and an inner hollow recess. Another object of the invention is to disclose the method as defined in any of the above, wherein finger-pad comprises a static portion and an outer dynamically active protruding circumference, interconnected by means selected from a group consisting a spring, sliding mechanism, rack and pinion mechanism, screw mechanism. pneumatic mechanism, motor, and any combination thereof. Another object of the invention is to disclose the method as defined in any of the above, wherein the elastic finger-pad is configured to deformed along the axis to a width W_{FPR}, equal or smaller than a function of the width of the epidermis W_{EPI}, so that W_{FPR}≤ϕ*W_{EPI}, about 0.25≤ϕ≥about 0.75. Another object of the invention is to disclose the method as defined in any of the above, wherein the elastic finger-pad is configured to deformed along the axis to a width W_{FPR}, equal or smaller than a function of the width of the stratum corneum W_{SC}, so that W_{SC}≤φ*W_{SC}, about 0.3≤φ≥ about 0.6. Another object of the invention is to disclose the method as defined in any of the above, wherein the elastic finger-pad is having an aperture in which a lancet is movable along the main longitudinal axis A:A

Another object of the invention is to discloses a method for piercing the body, comprising step of providing a body piercing device with a main longitudinal axis A:A; further providing the device a lancet reversibly movable in parallel with the axis from a first retract position, to a fully extended third position within the dermis and above and outside the venuole plexus, via a second position, located within the stratum cornea, and then back, to a final retracted (safely locked-) forth position; wherein the velocity of the lancet in its way from the first to the second position (V_{1st-2nd}) is relatively slow, namely V_{1st-2nd}≥ about 2 m/s. Another object of the invention is to disclose such a method wherein the acceleration of the lancet in its way from the first to the second position (A_{1st-2nd}) is relatively low, namely A_{1st-2nd}≥about 4 m/s². Another object of the invention is to disclose the method as defined in any of the above, wherein the device further characterized by an ergonomically contoured elastic finger-pad. Another object of the invention is to disclose the method as defined in any of the above, wherein the device is having a main longitudinal axis A:A, and the elastic finger-pad is configured to deform, narrow or otherwise retract along the axis. Another object of the invention is to disclose the method as defined in any of the above, wherein the finger-pad is a concaved member, having an outer elastic protruding circumference shaped by means of size and shape to snugly fit the finger pad, and an inner hollow recess. Another object of the invention is to disclose the method as defined in any of the above, wherein the elasticity of the finger-pad is ranging from Young's modulus: about 10³ to about 5*10⁹ Pa; from 0 to about 45Shore A; or 0 to about 80 Shore 00. Another object of the invention is to disclose the method as defined in any of the above, wherein the finger-pad is having an outer movable protruding circumference shaped by means of size and shape to snugly fit the finger pad, and an inner hollow recess. Another object of the invention is to disclose the method as defined in any of the above, wherein the finger-pad comprises a static portion and an outer dynamically active protruding circumference, interconnected by means selected from a group consisting a spring, sliding mechanism, rack and pinion mechanism, screw mechanism. pneumatic mechanism, motor, and any combination thereof. Another object of the invention is to disclose the method as defined in any of the above, wherein the elastic finger-pad is configured to deformed along the axis to a width W_{FPR}, equal or smaller than a function of the width of the epidermis W_{EPI}, so that W_{FPR}≤ϕ*W_{EPI}, about 0.25≤ϕ≥ about 0.75. Another object of the invention is to disclose the method as defined in any of the above, wherein the elastic finger-pad is configured to deformed along the axis to a width W_{FPR}, equal or smaller than a function of the width of the stratum corneum W_{SC}, so that W_{FPR}≤φ*W_{SC}, about 0.3≤φ≥ about 0.6.

In one aspect of the invention, an integrated diagnostic device for reducing or eliminating pain during body fluid sampling of a patient, comprising:
a. an enclosure forming a housing with inner walls, outer walls and a plurality of sub-units within said enclosure; said sub-units configured to complete an operation related to said body fluid sampling;
b. at least one pain reduction mechanism for alleviating pain associated with the penetrating of the skin by a penetrating member; and
c. a skin-penetrating mechanism comprising a penetrating member accommodated within said housing and configured to penetrate a skin of said patient.

In another aspect of the invention, the integrated diagnostic device above is provided, wherein said at least one pain reduction mechanism is a clamping mechanism, comprising:
a. a linear actuator extending from an upper or lower portion of said housing and moved by a patient finger; said linear actuator includes springs configured to facilitate linear motion, and open an adjustable jaw in an upper or lower portion of said housing, and ensure uniform clamping pressure;
b. an adjustable jaw configured to move along a linear path and apply a clamping force on a finger upon release of said linear actuator; and
c. an immobilizer on an upper side and finger padding on a lower side of said adjustable jaw configured to secure said finger in place, so that an upper cushion of said finger is aligned with said skin-penetrating mechanism.

In another aspect of the invention, the integrated diagnostic device as defined in any of above is provided, wherein said housing comprising:
a. an electrochemical sensor opening formed therein;
b. an electrochemical sensor pack having a sensor cavity, said sensor cavity being adapted to house a communicable test sensor therein;
c. a communicable test sensor being adapted to assist in the determination of an analyte concentration in the fluid sample;
d. a printed circuit board (PCB) disposed in said housing and adjacent to said communicable test sensor;
e. one or more members of a group consisting of power source, power-source adapter, power source input means; means for input communication, means for output communication and any combination thereof; and
f. optionally having an exterior with a grip.
   wherein said device is configured to (i) process electrical pulses obtained from said communicable test sensor, and (ii) communicate the processed digital data.

In another aspect of the invention, the integrated diagnostic device as defined in any of above is provided, wherein said skin penetrating mechanism comprising:
a. a lancet moveable between a standby position, an extended position, and a testing position;
b. a lancet mesh configured to store said lancet;
c. a lancet holder adapted to removably engage a base of said lancet,
d. a plunger coupled to said lance holder, said plunger having a central portion;
e. a shaft running through said central portion of the plunger, said plunger being adapted to move along said shaft, said shaft having an end portion that is adapted to secure said shaft to said integrated diagnostic device;
f. a spring at least partially surrounding said shaft, said spring being located between said plunger and said end portion of the shaft; and
g. a slider located on a rail on the interior of said housing, said slider being adapted to move along said rail in a first direction to compress said spring and wherein decompressing of said spring causes said plunger and said lance holder to rapidly move in a second direction opposite the first direction; and
h. a lancet button for the lancet to be released.

In another aspect of the invention, the integrated diagnostic device as defined in any of above is provided, wherein said at least one pain reduction mechanism is an ergonomically contoured elastic finger-pad; said ergonomically contoured elastic finger-pad is a concaved member, having an outer elastic protruding circumference shaped by means of size and shape to snugly fit said finger pad, and an inner hollow recess configured to accept the sharp end of said penetrating member.

In another aspect of the invention, the integrated diagnostic device as defined in any of above is provided, wherein size, shape, elasticity and materials of said ergonomically contoured elastic finger-pad is further configured to:
a. ensure, to either or both, (i) that body portion to be penetrated is provided in a tension so that it is held firmly and the skin is thin as possible, and (ii) said tension in effective to increase blood pressure in at least 5 mmHg as compared to local blood pressure before tensioning said body portion;
b. provide elasticity of said finger-pad ranging from Young's modulus: 10³ to 5*10⁹Pa; from 0 to 45 Shore A; or 0 to 80 Shore 00;
c. deform when pressed by a body portion to be pierced to a width W_{FPR}, equal or smaller than a function of the width of the epidermis W_{EPI}, so that W_{FPR}≤ϕ*W_{EPI}, 0.25≤ϕ≥0.75; and
d. deform when pressed by a body portion to be pierced to a width W_{FPR} , equal or smaller than a function of the width of the stratum corneum W_{SC}, so that c≤φ*W_{SC}, 0.3≤φ≥0.6.

In another aspect of the invention, the integrated diagnostic device as defined in any of above is provided, wherein said ergonomically contoured elastic finger-pad is further configured to one or more members of a group consisting of (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin. In another aspect of the invention, the integrated diagnostic device as defined in any of above is provided, wherein said skin penetrating mechanism comprising a lancet reversibly movable in parallel with a main longitudinal axis A:A of said device from a first retract position, to a fully extended third position within the dermis and above and outside the venuole plexus, via a second position, located within the stratum cornea, and then back, to a final retracted (safely locked-) fourth position; wherein the velocity of said lancet in its way from said first to said second position (V_{1st-2nd}) is relatively slow, namely V_{1st-2nd}≥2 m/s.

In another aspect of the invention, the integrated diagnostic device as defined in any of above is provided, wherein said housing accommodating:
a. a chip;
b. a lancet;
c. one or more analyte or analytes analyzing strips; and
d. one or more members of a group consisting of power source, power-source adapter, power source input means; means for input communication, means for output communication and any combination thereof.

In another aspect of the invention, the integrated diagnostic device as defined in any of above is provided, further comprising a wireless communication interface disposed within said housing and coupled to a controller, wherein said controller is adapted to wirelessly communicate with a remote device to provide a graphical user interface (GUI) for orchestrating a test of the performance metric of said body fluid and displaying results of said analysis.

In another aspect of the invention, the integrated diagnostic device as defined in any of above is provided, wherein said housing is a disposable member.

In one aspect of the invention, a body-piercing device for reducing or eliminating pain during piercing of the body, comprising:
a. an enclosure forming a housing with inner walls, outer walls and a plurality of sub-units within said enclosure; said sub-units configured to complete an operation related to said body fluid sampling;
b. at least one pain reduction mechanism for alleviating pain associated with the penetrating of the skin by a penetrating member;
c. a skin-penetrating mechanism comprising a penetrating member accommodated within said housing and configured to penetrate a skin of said patient.

In another aspect of the invention, the body-piercing device above is provided, wherein said at least one pain reduction mechanism is a clamping mechanism, comprising:
a. a linear actuator extending from an upper or lower portion of said housing and moved by a patient finger; said linear actuator includes springs configured to facilitate linear motion, and open an adjustable jaw in an upper or lower portion of said housing, and ensure uniform clamping pressure;
b. an adjustable jaw configured to move along a linear path and apply a clamping force on a finger upon release of said linear actuator; and
c. an immobilizer on an upper side and finger padding on a lower side of said adjustable jaw configured to secure said finger in place, so that an upper cushion of said finger is aligned with said skin-penetrating mechanism.

In another aspect of the invention, the body-piercing device as defined in any of above is provided, wherein said skin-penetrating mechanism comprising:
a. a lancet moveable between a standby position, an extended position, and a testing position;
b. a lancet mesh configured to store said lancet;
c. a lancet holder 128 adapted to removably engage a base of said lancet 112,
d. a plunger coupled to said lance holder 128, said plunger having a central portion;
e. a shaft running through said central portion of the plunger, said plunger being adapted to move along said shaft, said shaft having an end portion that is adapted to secure said shaft to said integrated diagnostic device;
f. a spring at least partially surrounding said shaft, said spring being located between said plunger and said end portion of the shaft; and
g. a slider located on a rail on the interior of said housing, said slider being adapted to move along said rail in a first direction to compress said spring and wherein decompressing of said spring causes said plunger and said lance holder to rapidly move in a second direction opposite the first direction; and
h. a lancet button for the lancet to be released.

In another aspect of the invention, the body-piercing device as defined in any of above is provided, further comprising an integrated diagnostic mechanism characterized by:
a. an electrochemical sensor opening formed therein;
b. an electrochemical sensor pack having a sensor cavity, said sensor cavity being adapted to house a communicable test sensor therein;
c. a communicable test sensor being adapted to assist in the determination of an analyte concentration in the fluid sample;
d. a printed circuit board (PCB) disposed in said housing and adjacent to said communicable test sensor; and
e. one or more members of a group consisting of power source, power-source adapter, power source input means; means for input communication, means for output communication and any combination thereof;
   wherein said body-piercing device is configured to (i) process electrical pulses obtained from said communicable test sensor, and (ii) communicate the processed digital data.

In another aspect of the invention, the body-piercing device as defined in any of above is provided, wherein said at least one pain reduction mechanism is an ergonomically contoured elastic finger-pad; said ergonomically contoured elastic finger-pad is a concaved member, having an outer elastic protruding circumference shaped by means of size and shape to snugly fit said finger pad, and an inner hollow recess configured to accept the sharp end of said penetrating member.

In another aspect of the invention, the body-piercing device as defined in any of above is provided, wherein size, shape, elasticity and materials of said ergonomically contoured elastic finger-pad is further configured to:
a. ensure, to either or both, (i) that body portion to be penetrated is provided in a tension so that it is held firmly and the skin is thin as possible, and (ii) said tension in effective to increase blood pressure in at least 5 mmHg as compared to local blood pressure before tensioning said body portion;
b. provide elasticity of said finger-pad ranging from Young's modulus: 10³ to 5*10⁹Pa; from 0 to 45 Shore A; or 0 to 80 Shore 00;
c. deform when pressed by a body portion to be pierced to a width W_{FPR}, equal or smaller than a function of the width of the epidermis W_{EPI}, so that W_{FPR}≤ϕ*W_{EPI}, 0.25≤ϕ≥0.75; and
d. deform when pressed by a body portion to be pierced to a width W_{FPR}, equal or smaller than a function of the width of the stratum corneum W_{SC}, so that c≤φ*W_{SC}, 0.3≤φ≥0.6.

In another aspect of the invention, the body-piercing device as defined in any of above is provided, wherein said ergonomically contoured elastic finger-pad is further configured to one or more members of a group consisting of (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin.

In another aspect of the invention, the body-piercing device as defined in any of above is provided, wherein said skin penetrating mechanism comprising a lancet reversibly movable in parallel with a main longitudinal axis A:A of said device from a first retract position, to a fully extended third position within the dermis and above and outside the venuole plexus, via a second position, located within the stratum cornea, and then back, to a final retracted (safely locked-) fourth position; wherein the velocity of said lancet in its way from said first to said second position (V_{1st-2nd}) is relatively slow, namely V_{1st-2nd}≥2 m/s.

In one aspect of the invention, a method for reducing or eliminating pain during body fluid sampling of a patient, comprising steps of:
a. obtaining the integrated diagnostic device as defined in any of the above;
b. actuating an adjustable jaw of a finger clamp along a linear path to open a fingertip aperture of said housing;
c. placing a finger on the lower side of said fingertip aperture on finger padding;
d. releasing said adjustable jaw to close said fingertip aperture and apply clamping pressure on said finger;
e. pressing a lancet button to release a lancet configured to pierce said finger through said finger clamp and to allow blood of said pricked finger accumulate on said fingertip;
f. actuating said adjustable jaw of said finger clamp to open said fingertip aperture;
g. retrieving said finger from said fingertip aperture;
h. rotating said finger to an electrochemical sensor opening of said housing;
i. placing said finger in a sensor cavity to apply a blood drop from said fingertip to a test strip therein; said test strip being adapted to assist in the determination of an analyte concentration in said fluid sample;
j. processing electrochemical signals from said test strip by a printed circuit board (PCB) disposed in said housing and adjacent to said test strip; and
k. communicating processed digital data.

In one aspect of the invention, an integrated diagnostic device with a clamping mechanism for reducing or eliminating pain during body fluid sampling, comprising:
a. an enclosure forming a housing with inner walls, outer walls and a plurality of sub-units within said enclosure; said sub-units configured to complete an operation related to said body fluid sampling;
b. a linear actuator extending from an upper portion of said housing and moved along a linear path by a thumb; said linear actuator configured to open an adjustable jaw in a lower portion of said housing along a linear path and apply a clamping force upon release; and
c. an adjustable jaw driven by said linear actuator and configured to move along a linear path to clamp a finger against an immobilizer and finger padding, so that a dorsal (upper) side of said finger is aligned with a retractable lancet.

In one aspect of the invention, an integrated diagnostic device for sample analysis, comprising:
a. a housing assembly comprising a rigid enclosure with a plurality of compartments, each designed to support different functional components configured to complete an operation of said system;
b. a linear actuator integrated into said housing assembly, configured to be manually controlled by a user; said linear actuator drives a movable clamping jaw along a predetermined linear path to apply a clamping force upon release;
c. a clamping unit comprising a moveable clamping jaw mounted on said linear actuator, designed to engage and securely hold a finger in place; said clamping unit further comprises a cushioned support and an immobilizer configured to ensure proper alignment of said finger with a lancet mechanism and create a unified pressure field on a dorsal (upper) side of said finger; and
d. a lancet mechanism configured to retract and release a lancet for pricking said finger; said lancet mechanism is activated in conjunction with said clamping unit to reduce pain and ensure accurate penetration and sample collection.

In one aspect of the invention, an integrated diagnostic device for analyzing a fluid sample, configured to:
a. linearly actuate an adjustable jaw of a finger clamp along a linear path to apply a clamping force to a finger to create a unified pressure field on a dorsal (upper) side of said finger, wherein a spring-loaded, lancet retraction mechanism is released to allow a lancet to prick said finger through said finger clamp and to allow blood of said pricked finger to contact a test strip after rotation of said finger;
b. analyze electrochemical signals output from said test strip coupled to a printed circuit board (PCB); and
c. generate analyte level data of said fluid sample, based upon said electrochemical signals, from which a performance metric may be determined.

In one aspect of the invention, an integrated diagnostic device with a clamping mechanism for reducing or eliminating pain during body fluid sampling, characterized by:
a. a housing optionally having an exterior with a grip, and an electrochemical sensor opening on its upper portion and formed therein; an electrochemical sensor pack having a sensor cavity, said sensor cavity being adapted to house a test sensor therein, said test sensor being adapted to assist in the determination of an analyte concentration in said fluid sample; a printed circuit board (PCB) disposed in said housing and adjacent to said test sensor;
b. a clamping mechanism comprising:
   i. a linear actuator extending from an upper portion of said housing and moved by a thumb; said linear actuator linear actuator comprising two shafts with a spring at least partially surrounding either shaft, configured to drive an adjustable jaw along a linear path to apply a clamping force upon release; and
   ii. an adjustable jaw configured to move along a linear path to clamp a finger against an immobilizer on an upper side and finger padding on a lower side, so that an upper side of said finger is aligned under said lancet and a unified pressure field is created on a dorsal (upper) side of said finger;
c. a lancing mechanism comprising:
   i. a lancet moveable between a standby position, an extended position, and a testing position;
   ii. a lancet mesh configured to store said lancet;
   iii. a lancet holder adapted to removably engage a base of said lancet,
   iv. a plunger coupled to said lance holder, said plunger having a central portion;
   v. a shaft running through said central portion of the plunger, said plunger being adapted to move along said shaft, said shaft having an end portion that is adapted to secure said shaft to said integrated diagnostic device;
   vi. a spring at least partially surrounding said shaft, said spring being located between said plunger and said end portion of the shaft; and
   vii. a slider located on a rail on the interior of said housing, said slider being adapted to move along said rail in a first direction to compress said spring and wherein decompressing of said spring causes said plunger and said lance holder to rapidly move in a second direction opposite the first direction;
   viii. a lancet button for the lancet to be released.

In another aspect of the invention, the integrated diagnostic device as defined in any of above is provided, wherein said housing has a size and a shape which allows for said housing to be grasped by a hand.

In another aspect of the invention, the integrated diagnostic device as defined in any of above is provided, wherein said housing is a disposable member.

In another aspect of the invention, the integrated diagnostic device as defined in any of above is provided, wherein said finger clamp is positioned and oriented on said housing to clamp a finger while said housing is grasped by said hand and said lancet is positioned and oriented to pierce skin from a dorsal (upper) side of said finger.

In another aspect of the invention, the integrated diagnostic device as defined in any of above is provided, further comprising a finger holster extending from the upper portion of said housing and positioned on said housing to secure a portion of a finger that has some blood for coupling to said test strip; said finger holster has a curved shape configured to cradle a side of said finger located opposite said test strip when said finger is positioned in said finger holster.

In another aspect of the invention, the integrated diagnostic device as defined in any of above is provided, wherein the linear actuator includes springs configured to facilitate linear motion and ensure uniform clamping pressure.

In another aspect of the invention, the integrated diagnostic device as defined in any of above is provided, wherein said adjustable jaw further comprises a leadscrew coupled to said linear actuator to translate a rotational force to a clamping force directed along said linear path, wherein said leadscrew extends into and engages with said adjustable jaw of said finger clamp.

In another aspect of the invention, the integrated diagnostic device as defined in any of above is provided, further comprising a wireless communication interface disposed within said housing and coupled to a controller, wherein said controller is adapted to wirelessly communicate with a remote device to provide a graphical user interface (GUI) for orchestrating a test of the performance metric of said body fluid and displaying results of said analysis.

In another aspect of the invention, the integrated diagnostic device as defined in any of above is provided, wherein said test strip is an electrochemical sensor.

In another aspect of the invention, the integrated diagnostic device as defined in any of above is provided, wherein said lancet button is a firing button located on a slider dock; said firing button being adapted to allow said spring to rapidly decompress when said firing button is depressed or moved.

In another aspect of the invention, the integrated diagnostic device as defined in any of above is provided, wherein said lancing mechanism further comprising an immobilizer configured to immobilize said finger in place; said immobilizer being adapted to regulate the distance said lancet can move in the direction of said finger.

In another aspect of the invention, the integrated diagnostic device as defined in any of above is provided, wherein said test strip is stored within said sensor cavity in said housing pack by enclosing said sensor cavity.

In another aspect of the invention, the integrated diagnostic device as defined in any of above is provided, wherein said PCB is adapted to electrochemically assist in the determination of an analyte concentration in said fluid sample and configured to process electrical pulses obtained from said test strip, and further to communicate processed digital data.

In another aspect of the invention, the integrated diagnostic device as defined in any of above is provided, wherein said integrated diagnostic device for analyte level testing is disposable.

In another aspect of the invention, the integrated diagnostic device as defined in any of above is provided, wherein said finger padding of said finger clamp is configured to ensure either or both:
a. that body portion to be pierced is provided in a tension so that it is held firmly and the skin is thin as possible; and
b. said tension is effective to increase blood pressure in at least 5 mmHg as compared to local blood pressure before tensioning said body portion.

In another aspect of the invention, the integrated diagnostic device as defined in any of above is provided, wherein said finger clamp is configured to one or more members of a group consisting of:
a. pressing skin region to be pierced, hence increasing sensation in piercing region before, whilst and somewhat after said lancet pierces said skin;
b. pressing said skin region to be pierced, hence decreasing skin's elasticity before and whilst lancet pierces said skin; and
c. pressing said skin region to be pierced, hence increasing local blood (capillary blood) pressure in said piercing region before and whilst lancet pierces said skin.

In another aspect of the invention, the integrated diagnostic device as defined in any of above is provided, wherein said lancet is reversibly movable from a first retract position, to a fully extended third position within the dermis and above and outside the venuole plexus, via a second position, located within the stratum cornea, and then back, to a final retracted (safely locked-) fourth position.

In another aspect of the invention, the integrated diagnostic device as defined in any of above is provided, wherein said housing further comprises sequential numbering configured to instruct a user how to analyze a fluid sample according to sequential steps.

In one aspect of the invention, a method for reducing or eliminating pain during body fluid sampling with an integrated diagnostic device, comprising steps of:
a. using a thumb-operated linear actuator to drive an adjustable jaw along a linear path, thereby opening a fingertip aperture;
b. positioning a finger within said fingertip aperture, ensuring said finger is aligned with an immobilizer on one side and cushioning padding on the opposite side;
c. releasing said linear actuator to close said fingertip aperture in said adjustable jaw, thereby applying a clamping force to secure said finger between said immobilizer and said padding;
d. ensuring that the upper side of the clamped finger is properly aligned with a retractable lancet integrated within said device;
e. releasing said lancet to prick said finger, allowing for the collection of a blood sample; and
f. collecting said blood sample from said finger to a collection area or test strip within said device for subsequent analysis.

In one aspect of the invention, a method for analyzing a fluid sample, comprising steps of:
a. providing an integrated diagnostic device within a housing having a clamping mechanism and a lancet;
b. providing said lancet with a fluid connection with a test strip;
c. configuring a printed circuit board (PCB) to process electrical pulses obtained from said test strip; and then
d. by means of said PCB, communicating processed digital data.

In one aspect of the invention, a method for reducing or eliminating pain during body fluid sampling with an integrated diagnostic device, comprising steps of:
a. obtaining the integrated diagnostic device of any one of claims 21 to 41;
b. actuating an adjustable jaw of a finger clamp along a linear path to open a fingertip aperture at the lower portion of a housing of said integrated diagnostic device;
c. placing a finger on the lower side of said fingertip aperture on finger padding;
d. releasing said adjustable jaw to close said fingertip aperture and apply clamping pressure against an immobilizer to create a unified pressure field on a dorsal (upper) side of said finger;
e. pressing a lancet button to release a lancet configured to prick said finger through said finger clamp and to allow blood of said pricked finger accumulate on the fingertip;
f. actuating said adjustable jaw of said finger clamp to open said fingertip aperture;
g. retrieving said finger from said fingertip aperture;
h. rotating said finger to an electrochemical sensor opening on the upper portion of said housing;
i. placing said finger in a sensor cavity to apply a blood drop from said fingertip to a test strip therein; said test strip being adapted to assist in the determination of an analyte concentration in said fluid sample;
j. processing electrochemical signals from said test strip by a printed circuit board (PCB) disposed in said housing and adjacent to said test strip; and
k. communicating processed digital data.

In another aspect of the invention, the method above is provided, wherein said housing has a size and a shape which allows for said housing to be grasped by a hand.

In another aspect of the invention, the method as defined in any of above is provided, wherein said housing is a disposable member.

In another aspect of the invention, the method as defined in any of above is provided, wherein said finger clamp is positioned and oriented on said housing to clamp a finger while said housing is grasped by said hand and said lancet is positioned and oriented to pierce skin from an upper side of said finger.

In another aspect of the invention, the method as defined in any of above is provided, further comprising a finger holster extending from the upper portion of said housing and positioned on said housing to secure a portion of a finger that has some blood for coupling to said test strip; said finger holster has a curved shape configured to cradle a side of said finger located opposite said test strip when said finger is positioned in said finger holster.

In another aspect of the invention, the method as defined in any of above is provided, wherein said adjustable jaw further comprises a leadscrew coupled to said linear actuator to translate a rotational force to a clamping force directed along said linear path, wherein said leadscrew extends into and engages with said adjustable jaw of said finger clamp.

In another aspect of the invention, the method as defined in any of above is provided, further comprising a wireless communication interface disposed within said housing and coupled to a controller, wherein said controller is adapted to wirelessly communicate with a remote device to provide a graphical user interface (GUI) for orchestrating a test of the performance metric of said body fluid and displaying results of said analysis.

In another aspect of the invention, the method as defined in any of above is provided, wherein said test strip is an electrochemical sensor.

In another aspect of the invention, the method as defined in any of above is provided, wherein said lancet button is a firing button located on a slider dock; said firing button being adapted to allow a spring to rapidly decompress or move when said firing button is activated.

In another aspect of the invention, the method as defined in any of above is provided, wherein said lancing mechanism further comprising an immobilizer configured to immobilize said finger in place; said immobilizer being adapted to regulate the distance said lancet can move in the direction of said finger.

In another aspect of the invention, the method as defined in any of above is provided, wherein said test strip is stored within a strip cavity in said housing pack by enclosing said strip cavity.

In another aspect of the invention, the method as defined in any of above is provided, wherein said PCB is adapted to electrochemically assist in the determination of an analyte concentration in said fluid sample and process electrical pulses obtained from said test strip, and further to communicate processed digital data.

In another aspect of the invention, the method as defined in any of above is provided, wherein said integrated diagnostic device is disposable.

In another aspect of the invention, the method as defined in any of above is provided, wherein said finger padding of said finger clamp is configured to ensure either or both:
a. that body portion to be pierced is provided in a tension so that it is held firmly and the skin is thin as possible; and
b. said tension is effective to increase blood pressure in at least 5 mmHg as compared to local blood pressure before tensioning said body portion.

In another aspect of the invention, the method as defined in any of above is provided, wherein said finger clamp is configured to one or more members of a group consisting of:
a. pressing skin region to be pierced, hence increasing sensation in piercing region before, whilst and somewhat after said lancet pierces said skin;
b. pressing said skin region to be pierced, hence decreasing skin's elasticity before and whilst lancet pierces said skin; and
c. pressing said skin region to be pierced, hence increasing local blood (capillary blood) pressure in said piercing region before and whilst lancet pierces said skin.

In another aspect of the invention, the method as defined in any of above is provided, wherein said lancet is reversibly movable from a first retract position, to a fully extended third position within the dermis and above and outside the venuole plexus, via a second position, located within the stratum cornea, and then back, to a final retracted (safely locked-) fourth position.

In another aspect of the invention, the method as defined in any of above is provided, wherein said housing further comprises sequential numbering configured to instruct a user how to analyze a fluid sample according to sequential steps.

In one aspect of the invention, a method for piercing the body, comprising step of providing a body piercing device with a clamping mechanism to create a unified pressure field on a dorsal (upper) side of a finger

### BRIEF DESCRIPTION OF THE DRAWINGS

The above and other features and advantages of the present invention will become more apparent to those of ordinary skill in the art by describing in detail exemplary embodiments thereof with reference to the attached drawings in which:

FIG. 1 is a front (a) and back (b) perspective view of an integrated diagnostic device for analyzing a fluid sample, according to a specific embodiment of the present invention;

FIG. 2 is a front perspective views of an integrated diagnostic device for analyzing a fluid sample, according to a specific embodiment of the present invention;

FIG. 3 is a front (a) and side (b) perspective view with length, width and height dimensions of an integrated diagnostic device for analyzing a fluid sample, according to a specific embodiment of the present invention;

FIG. 4 is a simplified illustration of the steps (features a-h) in analyzing a fluid sample by using an integrated diagnostic device, according to a specific embodiment of the present invention;

FIG. 5 is a front (a) and back (b) perspective view of an integrated diagnostic device for analyzing a fluid sample, according to another embodiment of the present invention;

FIG. 6 is a front (a) and side (b) perspective view with length, width and height dimensions of an integrated diagnostic device for analyzing a fluid sample, according to another embodiment of the present invention;

FIG. 7 is an exploded back perspective view of an integrated diagnostic device for analyzing a fluid sample, according to another embodiment of the present invention;

FIG. 8 is a simplified illustration of the steps (features a-d) in analyzing a fluid sample by using an integrated diagnostic device, according to another embodiment of the present invention;

FIG. 9 is a side perspective view of a finger to be pierced with an integrated diagnostic device for analyzing a fluid sample, according to another embodiment of the present invention;

FIG. 10 schematically illustrates a piercing device according to an exemplary embodiment of the present invention;

FIG. 11 is a simplified illustration of a cross-section of a body portion to be pierced with an integrated diagnostic device for analyzing a fluid sample, according to a specific embodiment of the present invention;

FIG. 12 schematically illustrates piercing mechanisms according to still another set of embodiments of the present invention;

FIG. 13 is a simplified illustration of a communicable system, according to a specific embodiment of the present invention; and

FIG. 14 is a schematic illustration of a chip, according to a specific embodiment of the present invention.

### DETAILED DESCRIPTION OF THE PREFERRED EMBODIMENTS

Exemplary embodiments of the present invention have been disclosed herein, and although specific terms are employed, they are used and are to be interpreted in a generic and descriptive sense only and not for purpose of limitation. Accordingly, it will be understood by those of ordinary skill in the art that various changes in form and details may be made without departing from the spirit and scope of the present invention as set forth in the following claims.

The terms “piercing device”, “finger pricker”, “lancet”, “injection device,” syringe” and “needle” as used herein refers to any minimal invasive medical device used to provide a way to take blood sample, e.g., by puncturing the body of a patient (e.g., user), and either provide a sample of body fluid (e.g., capillary blood), or to deliver medicament by way of parenteral administration. These injections include, but are not limited to intramuscular (IM), intravenous (IV), subcutaneous (SC), inravitreous, intraosseous infusion, intracerebral, intra-arterial, intracerebroventricular, intrathecal, among other injection types. The terms also refer to a small device narrowed at its outlet and fitted with either a piston or a rubber bulb for drawing in a quantity of fluid or for injecting fluid (e.g., insulin) into the body, whether made of glass, metal, or hard rubber. As used herein, the terms also refer to portable medical device narrowed at its outlet and fitted with either a piston or a rubber bulb for drawing in a quantity of fluid or for injecting fluid (e.g., insulin) into the body, whether made of e.g., glass, metal, or hard rubber.

Needle fear is a common barrier to initiating or adhering to medical treatments. Needle fear exists on a continuum of severity from dislike and discomfort to phobia. The terms “needle fear”, “needle anxiety” “needle phobia” and “trypanophobia” refer to anxiety associated with blood, puncturing and/or injections, as defined in the art, sec, e.g., Deacon, B., and J. Abramowitz. “Fear of needles and vasovagal reactions among phlebotomy patients.” Journal of anxiety disorders 20.7 (2006): 946-960.

The term “patient” as used herein refers broadly to subjects suspected of or known to be suffering from a disease or abnormality,

The term “analyte” designates without limitation a substance or chemical constituent (glucose, lipid, coagulation factor, hormone, inorganic material, such as sodium, magnesium etc., peptide, microorganism, drug, toxic material etc.) in a biological fluid (for example, blood, interstitial fluid, cerebral spinal fluid, lymph fluid or urine) that can be analyzed.

The term “blood” in the context of the invention encompasses whole blood and its cell-free components, such as plasma and serum. The term “capillary blood” refers to blood that is associated with any blood-carrying capillary of the body.

The terms “unified system” and “always unified system” interchangeably refer, in some sets of its embodiments, to a stand-alone platform, also known as an ‘application on chip’ (AOC) for providing an accurate, intuitively operated, yet not-expensive measurement of analyte(s). the term “system” also refers her for a system, device, module within the system or device, and a kit comprising said module, device and/or system. In some of the embodiments the system comprises a lancet and actuation mechanism, system-on-Chip (SoC), ASIC, and/or AOC, and analyzing means, e.g., chemical strip. In some of the embodiments the system comprises the same, and IoT, communication means etc. In some of the embodiments the system comprises the same, and finger-pad for one or more of the followings: (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin. This disposable and communicable testing unified system allows people to test themselves before first symptoms of a disease, hence the system avoids the diseases before its occurrence or its manifestation.

The term “measurement” may refer to a signal that is indicative of a concentration of an analyte in a medium, such as a current signal, for example, to a more typical indication of a concentration of an analyte in a medium, such as mass of the analyte per unit volume of the medium, for example, or the like. The term “value” may sometimes be used herein as a term that encompasses the term “measurement.”

The term “concentration” may refer to a signal that is indicative of a concentration of an analyte in a medium, such as a current signal, for example, to a more typical indication of a concentration of an analyte in a medium, such as mass of the analyte per unit volume of the medium, for example, or the like

The term “biological sample,” as used herein is a broad term, and is to be given its ordinary and customary meaning to a person of ordinary skill in the art (and is not to be limited to a special or customized meaning), and refers without limitation to sample of a host body, for example, blood, interstitial fluid, spinal fluid, saliva, urine, tears, sweat, or the like.

As used herein, the term “pain” is given its broadest meaning and includes an unpleasant sensation or emotional experience associated with actual tissue damage or potential tissue damage, or with respect to such damage. The term also refers to all types of pain, including somatic pain, e.g., visceral pain or cutaneous pain, or pain caused by a burn, a bruise, an abrasion, a laceration, a broken bone, a torn ligament, a torn tendon, a torn muscle etc.

The terms “substantially” “about” and “approximately” as interchangeably used herein is a broad term, and is to be given its ordinary and customary meaning to a person of ordinary skill in the art (and is not to be limited to a special or customized meaning), and refers without limitation to being largely but not necessarily wholly that which is specified, which may include an amount greater than 50 percent, an amount greater than 60 percent, an amount greater than 70 percent, an amount greater than 80 percent, an amount greater than 90 percent or more.

The terms “processor” and “processor module,” as used herein are a broad terms, and are to be given their ordinary and customary meaning to a person of ordinary skill in the art (and are not to be limited to a special or customized meaning), and refer without limitation to a computer system, state machine, processor, or the like designed to perform arithmetic or logic operations using logic circuitry that responds to and processes the basic instructions that drive a computer. In some embodiments, the terms can include ROM and/or RAM associated therewith.

The term “ROM,” as used herein is a broad term, and is to be given its ordinary and customary meaning to a person of ordinary skill in the art (and is not to be limited to a special or customized meaning), and refers without limitation to read-only memory, which is a type of data storage device manufactured with fixed contents.

The term “condition,” as used herein is a broad term, and is to be given its ordinary and customary meaning to a person of ordinary skill in the art (and is not to be limited to a special or customized meaning), and refers without limitation to a mode or state of being; the physical status of the body as a whole or of one of its parts. For example, a host's condition can refer to his state of health, his metabolic state and the like.

The term “glucose boundary,” as used herein is a broad term, and is to be given its ordinary and customary meaning to a person of ordinary skill in the art (and is not to be limited to a special or customized meaning), and refers without limitation to a glucose concentration or range of glucose concentrations. In some embodiments, the system is configured to compare and/or evaluate internally derived data with a glucose boundary. In some embodiments, a glucose boundary can include a maximum glucose concentration.

The term “chip” as used herein, refers to an integrated circuit or monolithic integrated circuit, also referred to as an IC, or a microchip, is a set of electronic circuits on one small flat piece of semiconductor material, e.g., silicon. in an embodiment of the invention, large numbers of small metal-oxide-semiconductor field-effect transistors integrate into a single chip. as used hereinafter, chip is in size ranging, from 10 nm or less, to 200 nm or more, e.g., between about 20 nm to about 175 nm. The term also denoted for an application-specific integrated circuit. The term also denoted for an electrical or digital means comprising or interconnected with an application-specific instruction set processor (ASIP).

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it covers all modifications and alternatives coming within the true scope and spirit of the invention.

The term “elasticity” refers to the ability of a deformed material body to return to its original shape and size when the forces causing the deformation are removed. Otherwise, the term refers to the ability of a body to resist a distorting influence and to return to its original size and shape when that influence or force is removed. It well in the scope of the invention wherein Young's modulus (E), which describes tensile elasticity, or the tendency of an object to deform along an axis when opposing forces are applied along that axis (the ratio of tensile stress to tensile strain) is ranging e.g., from about 10³ to about 5*10⁹Pa; a range of 0 to about 45 Shore A; or otherwise 0 to about 80 Shore 00.

The integrated diagnostic device for analyzing a fluid sample of the present disclosure, and its variations, is novel and inventive by enabling several significant features not provided by any reasonable understanding of the prior art or combination thereof. Firstly, the device includes a clamping mechanism which is configured to immobilize a finger, such that it is fixated firmly between an immobilizer and finger padding components. The clamping mechanism is configured to apply a clamping force on a finger to be sampled, thereby creating a “bubble” in the finger pad of the immobilized finger. The “bubble” to be pierced is provided in a tension so that it is held firmly and the skin is thin as possible, thus reducing pain when pierced by the retractable lancet. The tension is also effective to increase blood pressure in at least 5 mmHg as compared to local blood pressure before tensioning the upper pad of the finger and said bubble. Secondly, the lancet mechanism is not user-dependent, but rather executed by a predefined spring mechanism, with constant speed, velocity and retractable stages. The spring-dependent lancet mechanism allows for a one-time sampling event and prevents the necessity of resampling.

The clamping mechanism plays a critical role in ensuring accurate, consistent, and efficient blood sample collection in the integrated diagnostic device. Its primary purpose is to stabilize the finger during the sampling process and create the optimal conditions for blood extraction, by applying controlled pressure to increase local blood flow and reduce the risk of movement during the lancing procedure.

The clamping mechanism in the integrated diagnostic device plays a crucial role in reducing pain during the finger-piercing process. By immobilizing the finger securely between the adjustable jaw and the fixed immobilizer, the device creates a stable environment that minimizes any involuntary movements. This stability is essential, as it allows for precise lancet placement, ensuring that the puncture is accurate and controlled. Furthermore, the gentle compression applied by the mechanism creates a “bubble” effect in the finger pad, increasing local blood flow while maintaining a thinner skin surface at the puncture site. This hemodynamic effect helps to reduce the pain experienced during the lancing, as a well-prepared site allows for a more effective and less traumatic penetration.

Additionally, the soft padding integrated into the clamping mechanism enhances user comfort by providing a gentle grip on the finger. This padding not only protects the skin but also prevents excessive pressure that could lead to bruising or discomfort. By carefully controlling the clamping force, the device reduces the depth of penetration required by the lancet, further minimizing pain. Potential feedback mechanisms, including tactile and auditory signals, inform users when the finger is optimally secured, adding to their confidence and reducing anxiety associated with the lancing process. Together, these features ensure a more comfortable experience, making blood sampling easier and less distressing for users.

The clamping mechanism consists of several key components, all housed within the device's outer casing. These include: 1) Adjustable Jaw: a movable, adjustable jaw is responsible for securing the user's finger in place. This jaw moves along a predetermined linear path, driven by a linear actuator, which is controlled manually via a thumb-operated slider or electronically through an internal motor; 2) Immobilizer: on the opposite side of the adjustable jaw is a fixed or semi-fixed immobilizer. The immobilizer presses against the dorsal (upper) side of the finger, acting as a cushion to protect the user's skin while preventing movement during the procedure; 3) Padding: to ensure user comfort, the inner surfaces of both the adjustable jaw and immobilizer are lined with soft padding. This padding provides a secure, but gentle, grip on the finger, reducing discomfort and preventing excessive pressure that could cause bruising; and 4) Linear Actuator: the linear actuator enables precise movement of the adjustable jaw. It may be manually operated by a thumb slider or lever that drives the jaw forward and backward. In more advanced versions, the actuator could be powered by a small motor controlled by an onboard processor, allowing for automatic adjustments based on the user's finger size.

In terms of operation of the clamping mechanism, it is designed to operate in a seamless, user-friendly manner. The mechanism works in the following step-by-step manner: 1) Finger Placement: the user inserts their finger into a finger “cradle” or finger padding located between the adjustable jaw and the immobilizer. The finger padding helps guide the finger into the correct position, ensuring that the sampling area (usually the fingertip or the side of the finger) aligns with the lancet assembly; 2) Clamping Activation: once the finger is positioned correctly, the user activates the clamping mechanism. This can be done by sliding a thumb-operated lever or pressing a button, depending on the device's design. The linear actuator moves the adjustable jaw towards the immobilizer, applying gentle, even pressure to the finger; 3) Controlled Compression: as the adjustable jaw tightens, it creates controlled compression on the skin. This compression serves two main functions: a) Immobilization: by holding the finger securely in place, the clamping mechanism prevents any involuntary movements during the lancing procedure, ensuring that the lancet hits the target location with precision, and b) Increased Blood Flow: the slight pressure applied by the clamping mechanism enhances local blood flow by compressing the surrounding tissue and temporarily increasing the blood pressure in the area by at least 5 mmHg. This hemodynamic effect is crucial for ensuring that a sufficient sample of blood can be collected with a single lancet prick; 4) Feedback Mechanism: to enhance user confidence and improve accuracy, the device may include a feedback mechanism. This could take the form of: a) Tactile Feedback: a click or vibration when the correct clamping pressure is reached; b) Visual Feedback: a LED indicator or digital display that turns green when the finger is clamped securely and is ready for lancing; and c) Auditory Feedback: a soft beep or chime that signals the user to proceed with the next step; 5) Lancing and Blood Collection: once the finger is clamped securely, the user activates the lancet mechanism to pierce the skin and collect the blood sample. The controlled pressure exerted by the clamping mechanism ensures that the puncture is consistent and effective, allowing blood to pool at the puncture site for easy collection; and 6) Release: after the sample is collected, the user deactivates the clamping mechanism by releasing the thumb slider.

As an integral part of the integrated diagnostic device, the clamping mechanism provides: 1) Consistency in Sampling: the clamping mechanism guarantees consistent placement and immobilization of the finger, reducing variability in lancet penetration depth and blood collection. This leads to more reliable test results across repeated uses; 2) Improved Blood Flow: by applying controlled pressure, the clamping mechanism increases blood perfusion to the puncture site. This allows for faster and more efficient blood collection, particularly for individuals with poor circulation or for tests that require larger blood samples; 3) Reduced Pain and Discomfort: the soft padding on the jaw and immobilizer provides comfort during the clamping process. Additionally, the mechanism's controlled compression minimizes the depth of penetration required by the lancet, reducing the pain associated with lancing; 4) Safety and Precision: immobilizing the finger reduces the risk of lateral movement or jerking, which could cause accidental injury or inaccurate lancing. The feedback system ensures that the user knows when the device is correctly positioned and ready for use; and 5) Ease of Use: the simple thumb-operated design makes the clamping mechanism intuitive to use, even for individuals with limited dexterity. This is especially beneficial for elderly patients or those with conditions like arthritis.

In some embodiments of the device, the clamping mechanism may feature advanced automation and adaptability, as follows: 1) Automated Clamping: instead of manual operation, the clamping mechanism could be fully automated, with the adjustable jaw moving into place once the device detects a finger has been inserted. Sensors could monitor the amount of pressure being applied and adjust accordingly to optimize blood flow without causing discomfort; 2) Adjustable Pressure Settings: for users with specific medical conditions, such as peripheral artery disease or diabetes, where circulation may be impaired, the device could feature adjustable pressure settings. This would allow for more or less compression based on individual needs; and 3) Smart Sensors: Integrated sensors could detect the thickness of the user's finger and automatically adjust the clamping pressure to match, providing a more personalized and effective experience.

In some embodiments, the clamping mechanism may include a manually operated actuator (e.g., a lever or dial) that allows the user to adjust the clamping force. The force applied by the immobilizer and finger padding can be increased or decreased based on the user's preference or specific physiological needs (e.g., sensitive skin, reduced blood flow). The manual adjustability provides fine control over the creation of the “bubble” on the finger pad, ensuring optimal tension in the skin for efficient blood sampling.

In another embodiment, the clamping mechanism may be fully automated. The device is equipped with smart sensors that detect the size, thickness, and temperature of the user's finger. The clamping force is automatically adjusted to ensure the correct tension is applied to the finger pad, creating the “bubble” under ideal conditions. This automated system ensures consistency and reduces user error. Additionally, the smart sensors could provide real-time feedback on blood pressure in the clamped area, ensuring that the desired increase in local blood pressure (e.g., by 5 mmHg) is achieved before lancing.

In some embodiments, the clamping mechanism may be designed with multi-position functionality, allowing it to adjust to different areas of the finger. For example, the mechanism could allow sampling from the side of the finger, where the skin is thinner and often less painful to pierce, or from the center of the pad, where more blood flow may be present. This flexibility provides users with options to choose the most comfortable and efficient sampling location. In further embodiments, the clamping mechanism may include dynamic tension control by using a microprocessor-controlled actuator to gradually increase or decrease the clamping pressure in response to real-time feedback. The device could monitor skin tension and blood flow beneath the immobilized finger pad, adjusting the clamping force dynamically to maintain the ideal conditions for a painless, efficient blood sample.

In some embodiments, the lancet mechanism involves a spring-assisted lancet with an adjustable speed and depth of penetration. The user may select from predefined settings that modify the speed of the lancet based on their sensitivity or blood flow characteristics. For example, the device may include multiple preset modes, such as “Sensitive Mode” for users with delicate skin and “High Flow Mode” for users with reduced circulation. These adjustments optimize the sampling process to avoid unnecessary pain while ensuring sufficient blood collection.

In another embodiment, the lancet mechanism includes a predefined safety stop that ensures the lancet cannot penetrate too deeply. This prevents the lancet from causing excessive injury or discomfort, particularly for users with thin or delicate skin. The constant velocity of the lancet's movement ensures consistent results across multiple uses, reducing the need for resampling due to insufficient blood collection.

In yet another embodiment, the lancet mechanism could feature a multi-lancet cartridge system. This system allows for multiple lancets to be loaded into the device at once, enabling consecutive blood sampling without the need for reloading the lancet after each use. The multi-lancet system could be particularly beneficial in clinical settings where rapid testing of multiple patients is required, or for users who need frequent blood monitoring.

In further embodiments, the device includes an automated lancet mechanism with integrated blood flow detection sensors. After the clamping mechanism creates the “bubble” within the finger pad of the user, and the tension in the skin is optimized, the lancet is deployed automatically once the device detects sufficient blood flow beneath the skin. This feature ensures that the lancet only activates when the blood sample is likely to be adequate, reducing the chances of failed lancing attempts and preventing unnecessary repeat samplings.

In another embodiment, the lancet mechanism includes a micromotor-controlled lancet. Instead of relying solely on spring tension, the lancet is driven by a micromotor that ensures absolute control over the speed, depth, and retraction of the lancet. This mechanism allows for precision lancing, particularly useful for users with conditions that affect blood clotting or skin integrity, as it allows for precise, shallow penetrations with minimal trauma.

One embodiment of the device integrates a pre-lancing blood pressure monitoring system within the clamping mechanism. The device is equipped with sensors that measure local blood pressure in the finger before and after clamping. The clamping force is then adjusted to ensure that the increase in local blood pressure (e.g., at least 5 mmHg) is achieved before the lancet is triggered. This ensures optimal conditions for blood sampling and reduces the need for multiple lancing attempts.

Another embodiment involves the device incorporating temperature sensors to adjust the clamping force and tension based on the user's skin temperature. Warmer skin typically has better blood flow, while cooler skin may require additional tension to achieve sufficient blood pooling. The device dynamically adjusts to the user's temperature, optimizing the formation of the “bubble” within the finger pad of the user, and ensuring efficient blood extraction.

In a further embodiment, the clamping mechanism includes a timed pressure release system. After the lancet pierces the skin, the clamping force is briefly increased and then gradually released over a few seconds, promoting enhanced blood flow to the puncture site. This technique reduces the chance of incomplete blood samples and improves the overall efficiency of the sampling process. As an example, the system may be provided as a-or as a part of a device, utilizable as a glucometer, for testing (capillary) blood glucose concentration: it is a small and portable, ready-to-use, single-use, disposable glucometer characterized by both non-tedious and intuitively-operated, free of pain and anxiety for pain or discomfort, and improved user experience.

In some embodiments, a glucometer in an elongated device having a main longitudinal axis A:A, in one embodiment of the invention, lateral cross-section (perpendicular to axis A:A) of the device is circular or oval. Alternatively, at least a portion of the cross section is polygonal (not shown). The glucometer comprises, inter alia, a head portion, a body portion and analyzing strip (e.g., diabetes test strip30). Various diabetes test strips are commercially available, including e.g., Accu-Chek Aviva Plus™ test strips or Accu-Chek SmartView™ test strips, by Roche Diabetes Care, Inc. (US); FreeStyle™ Abbott Precision Xtra™ Strips, by Abbott (US); GE200™ Test Strips by GE (US).

In some embodiments, the head portion may comprise gripping protrusions enabling rotation of the head portion around the main axis relatively to the body. Rotation of the head may facilitate or otherwise enable actuation of a lancet (not shown, positioned, e.g., fleshy underside of the end of a body portion to be pierced, e.g., a finger pad). The lancet is accommodated within portions of the head and/or body, and positioned in parallel with the main axis, e.g., in a concentric or eccentric manner. As the head is potentially rotatable, indication means are provided. illustration 100A shows the same. Head portion comprises a finger pad having an inner embedded portion (2), configured to accept the activated lancet (not shown), and a protruded circumference.

It is well in the scope of the invention where medical device, system. Module or kit 100, also denoted as SmartStrip™, Smart-Lancet™, and/or SmarTest™, may be useful for one or more of the following: (i) collecting analytes and biological samples, (ii) measuring the same so that it is characterized quantitatively vs quantitively to define a value(s) or range thereof, concentration(s) or range thereof; and further (iii) processing/translating by a processor and/or communicating the result(s) to a database, ROM etc., so that the current condition (e.g., in light of a baseline) of the user is obtained. In case of a glucometer, device 100 samples, tests, presents user's current glucose level (condition) in light of glucose boundaries.

It is in the scope of one set of embodiments of the invention wherein at least one portion of circumference 1 is finger-pad is one or more of the following: elastic, configured to deform, narrow or otherwise retract along the axis. Additionally, or alternatively, the elasticity of the finger-pad is characterized by a Young's modulus ranging from any of the following: about 10³ to about 5*10⁹ Pa; from about 10³ to about 10⁸ Pa; from about 10² to about 10⁹ Pa; or from about 10² to about 10⁸ Pa.

It is in the scope of the invention wherein the elastic finger-pad is made of, or otherwise comprises polymers having Young modulus (MPa) and elongation (%) such as polyethylene LDPE 172-282, 100-650; polypropylene 1138-1551, 100-600; polyurethane 0.17-34.47, 250-800; polyethylene terephthalate 2578-4137, 30-300; polyamide (PA66) 1586-3447, 150-300; rubbers, silicone, and any mixture thereof.

The Shore 00 Hardness Scale measures rubbers and gels that are very soft. The Shore A Hardness Scale measures the hardness of flexible mold rubbers that range in hardness from very soft and flexible, to medium and somewhat flexible etc. Hence, additionally, or alternatively to the defined above, the elasticity of the finger-pad is characterized by Shore A ranging from any of the following: 0 to about 30; from 0 to about 40; from 0 to about 50; from about 10 to about 30 or from about 10 to about 45 Shore A; Additionally, or alternatively to the defined above, the elasticity of the finger-pad is characterized by Shore 00 ranging from any of the following: from 0 to about 80;from about 10 to about 70; from about 20 to 6about 0; or from about 20 to about 80 Shore 00.The finger-pad ensures, by means of its size, shape, elasticity and materials, that the skin to be pierced is provided in a tension so that it is held firmly and the skin is thin as possible. This method of providing the skin to be pierced in tension reduces both pain and fear-of-pain.

It is still in the scope of this set of embodiments wherein the finger-pad is having an outer movable protruding circumference shaped by means of size and shape to snugly fit the body portion to be pierced, e.g., a finger pad, and an inner hollow recess.

Additionally, or alternatively, the finger-pad comprises a static portion and an outer dynamically active protruding circumference, interconnected by means selected from a group consisting e.g., a spring, a sliding mechanism, a rack and pinion mechanism, a screw mechanism. a pneumatic mechanism, a motor, and any combination thereof.

It is also in the scope of the invention wherein finger-pad according to one set of embodiments, is configured by e.g., its size, shape, texture, composition, elasticity, movability and mechanism of action, to reduce skin elasticity before piercing. It is also in the scope of the invention wherein the finger-pad, according to yet another set of embodiments, allows the lancet to pierce the skin in low momentum, and/or low impulse (J, integral of a force over time).

It is further in the scope of the invention wherein finger-pad according to one set of embodiments, is configured by e.g., its size, shape, texture, composition, elasticity, movability and mechanism of action, to press the skin and thus to increase sensation in the piercing region before, whilst and somewhat after lancet pierces the skin.

It is further in the scope of the invention wherein finger-pad according to one set of embodiments, is configured by e.g., its size, shape, texture, composition, elasticity, movability and mechanism of action, to press the skin and thus to increase local blood (capillary blood-) pressure in the piercing region before and whilst lancet.

It is further in the scope of the invention wherein finger-pad according to one set of embodiments, is configured by e.g., its size, shape, texture, composition, elasticity, movability and mechanism of action, to one or more member of a group consisting of (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin.

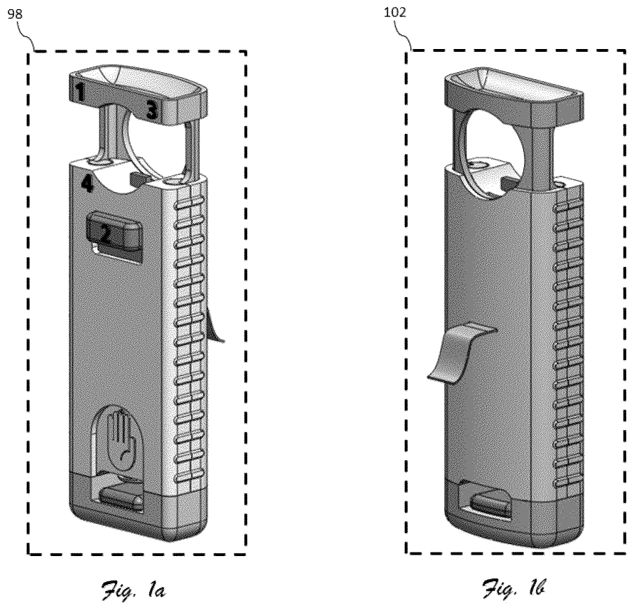

Reference is now made to FIGS. 1a and 1b, illustrating in both a non-limiting and in out of scale manners, a front 98 and back 102 views, respectively, of a medical device useful for extracting a body fluid sample for further analyses in order to make a diagnosis or to monitor the concentration of analytes, according to one embodiment of the invention. This device is provided useful as a glucometer, for testing (capillary) blood glucose concentration: it is a small and portable, ready-to-use, single-use, disposable glucometer characterized by both non-tedious intuitively-operated clamping mechanism, free of pain and anxiety for pain or discomfort, and improved user experience.

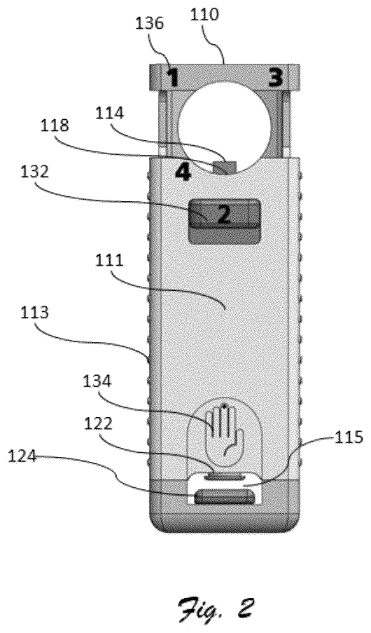

FIG. 2 illustrates a front perspective view of an integrated diagnostic device for analyzing a fluid sample, according to a specific embodiment of the present invention. Diagnostic device in one embodiment includes a housing 111 having an exterior with a grip 113, and an electrochemical sensor opening 118 on its upper portion and formed therein, and an electrochemical sensor pack having a sensor cavity adapted to house a test sensor 114 therein. The test sensor 114 being adapted to assist in the determination of an analyte concentration in the fluid sample. The clamping mechanism comprises a) a linear actuator extending from an upper portion of the housing and moved by a thumb; said linear actuator 110 configured to drive an adjustable jaw 115 along a linear path to apply a clamping force; and b) an adjustable jaw 115 configured to move along a linear path to clamp a finger against an immobilizer 122 on an upper side and finger padding 124 on a lower side, so that an upper side of the finger 120 is aligned under the lancet. The diagnostic device includes a lancing mechanism with a retractable lancet and a lancet button 132 for the lancet to be released. An orientation icon 134 indicates that the user must use a middle finger to be placed in the immobilizer.

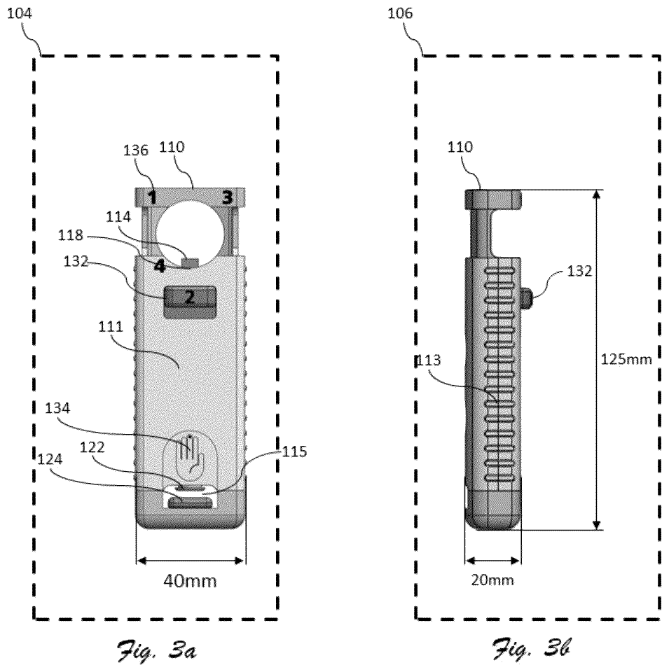

Reference is now made to FIGS. 3a and 3b, illustrating a front 104 and side 106 perspective views, respectively, of an integrated diagnostic device for analyzing a fluid sample, according to a specific embodiment of the present invention. Diagnostic device in one embodiment includes a housing 111 with dimensions of 40 mm length, 20 mm width and 125 mm height. The housing having an exterior with a grip 113, and an electrochemical sensor opening 118 on its upper portion and formed therein, and an electrochemical sensor pack having a sensor cavity adapted to house a test sensor 114 therein. The test sensor 114 being adapted to assist in the determination of an analyte concentration in the fluid sample. The clamping mechanism comprises a) a linear actuator extending from an upper portion of the housing and moved by a thumb; said linear actuator 110 configured to drive an adjustable jaw 115 along a linear path to apply a clamping force; and b) an adjustable jaw 115 configured to move along a linear path to clamp a finger against an immobilizer 122 on an upper side and finger padding 124 on a lower side, so that an upper side of the finger 120 is aligned under the lancet. The diagnostic device includes a lancing mechanism with a retractable lancet and a lancet button 132 for the lancet to be released. An orientation icon 134 indicates that the user must use a middle finger to be placed in the immobilizer.

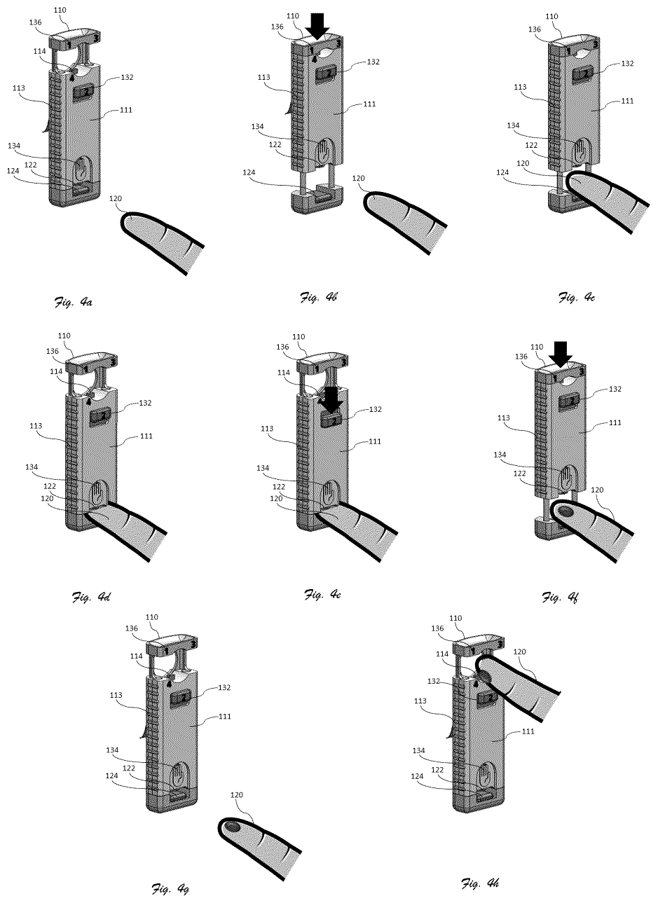

Reference is now made to FIG. 4 illustrating a simplified illustration of the steps (a-h) in analyzing a fluid sample by using an integrated diagnostic device, according to a specific embodiment of the present invention. The work flow begins (FIG. 4a) when a user presents a finger 120 with the finger pad facing upwards, wherein the device is in stationary position with the adjustable jaw 115 of the clamping mechanism closed. The user places a thumb on the top of the linear actuator 110, pressing down, upon which the adjustable jaw 115 opens, and a gap is created between the immobilizer 122 and finger padding 124 (FIG. 4b). While still pressing the thumb down on the linear actuator 110 and maintaining the gap open, the user may place a finger 120 from the second hand on the finger padding 124, so that the finger pad is facing up (FIG. 4c). Once positioned correctly, the user may release the thumb pressing the linear actuator 110, thereby moving the adjustable jaw 115 downwards and closing the gap so that the immobilizer 122 clamps down on the finger pad (FIG. 4d). The finger 120 to be sampled is firmly fixed between the immobilizer 122 and the finger padding 124, when the user may press a lancet button 132 for the lancet to be released, wherein the retractable lancet pierces the skin of the finger pad clamped between the immobilizer 122 and finger padding 124 (FIG. 4c). The user then again places a thumb on the top of the linear actuator, pressing down, upon which the adjustable jaw 115 opens, and the user may remove the pierced finger 120 from the gap between the immobilizer 122 and finger padding 124 (FIG. 4f), after which the user may release the thumb pressing down the linear actuator 115 and closing the adjustable jaw 115 (FIG. 4g). Lastly, the user rotates the finger 120 to an electrochemical sensor 114 opening on upper portion of the device housing 111. The finger 120 is placed in a sensor cavity to apply a blood drop from the fingertip to a test strip 114, wherein the test strip 114 being adapted to assist in the determination of an analyte concentration in the blood sample by processing electrochemical signals from the test strip 114 by a printed circuit board (PCB) 116 disposed in the housing 111 and adjacent to said test strip 114.

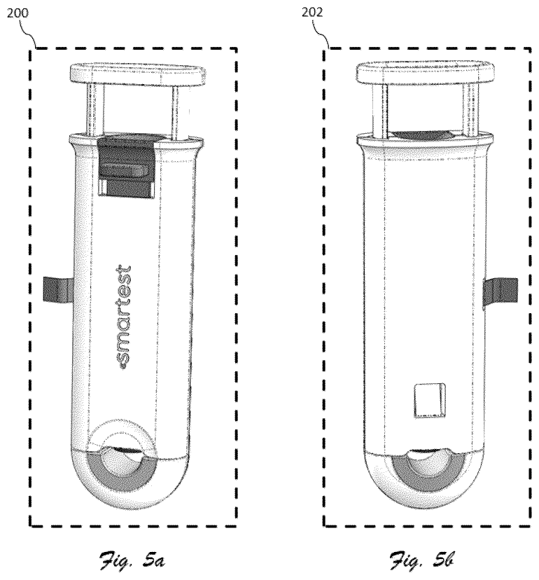

FIGS. 5a and 5b shows a front 200 and back 202 perspective view, respectively, of an integrated diagnostic device for analyzing a fluid sample, according to another embodiment of the present invention. The device is compact, portable, and ready for single-use as a disposable glucometer. The device features an easy-to-use, intuitively operated clamping mechanism that minimizes pain and discomfort, eliminating anxiety related to the sampling process, while offering an enhanced user experience.

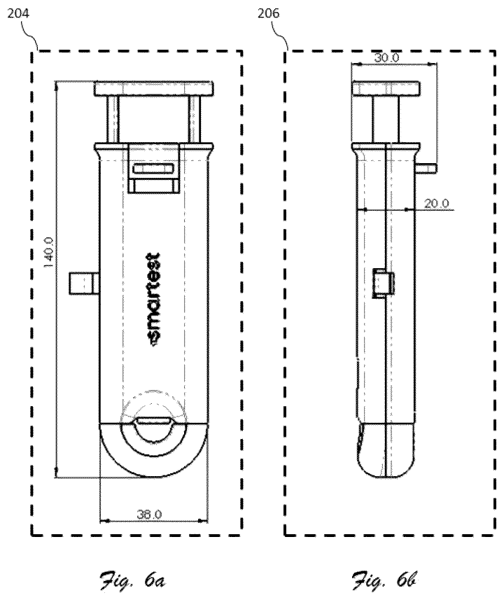

FIGS. 6a and 6b, illustrate front 204 and side 206 perspective views, respectively, of an integrated diagnostic device for analyzing a fluid sample, according to a specific embodiment of the present invention. The diagnostic device in one embodiment includes a housing with dimensions of 36 mm length, 20 mm width and 140 mm height.

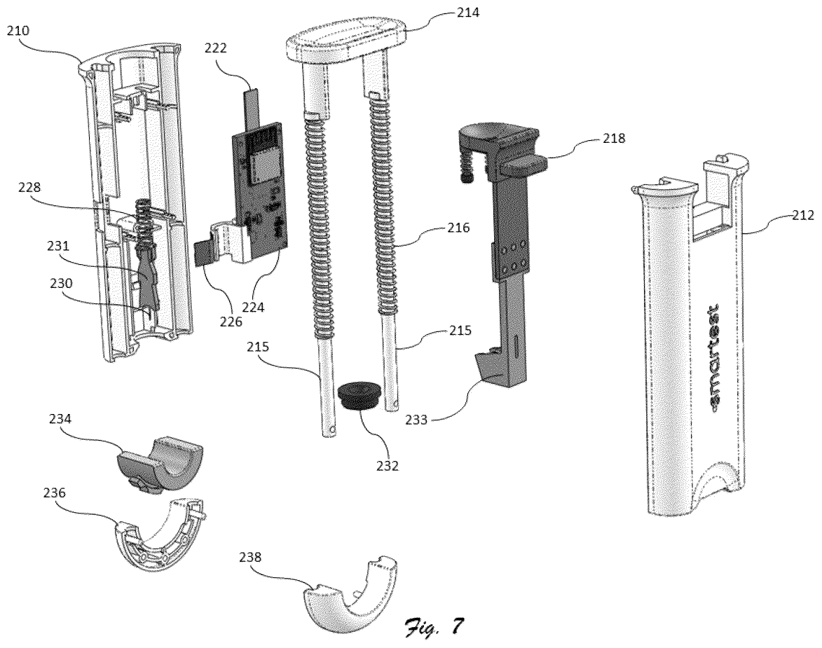

Reference is now made to FIG. 7 illustrating an exploded perspective view of an integrated diagnostic device for analyzing a fluid sample, according to another embodiment of the present invention. Diagnostic device in one embodiment includes a front 210 and rear 212 covers of the housing, and an electrochemical sensor opening on its upper portion and formed therein, and an electrochemical sensor pack having a sensor cavity adapted to house a test sensor 222 therein. The test sensor 222 being adapted to assist in the determination of an analyte concentration in the fluid sample. The clamping mechanism comprises a) a linear actuator 214 comprising two shafts 215 with a spring 216 at least partially surrounding either shaft 215, on either shaft extending from an upper portion of the housing and moved by a thumb; said linear actuator 214 configured to drive an adjustable jaw along a linear path to apply a clamping force; and b) an adjustable jaw configured to move along a linear path to clamp a finger against an immobilizer or finger mount 232 on an upper side and finger padding 234 on a lower side, so a finger pad is aligned under the lancet 230. The immobilizer or finger mount 232 may also serve as a lancet needle guide. The finger padding is comprised of a front 236 and rear 238 finger pad holder. The diagnostic device includes a lancing mechanism comprising a) a lancet 230 moveable between a standby position, an extended position, and a testing position, b) a lancet mesh 231 configured to store the lancet 230, c) a lancet holder 233 adapted to removably engage a base of the lancet 230, d) a plunger, having a central portion, coupled to the lancet holder 233, c) a shaft running through the central portion of the plunger, being adapted to move along the shaft; said shaft having an end portion that is adapted to secure the shaft to the integrated diagnostic device, f) a spring 228 at least partially surrounding the shaft, being located between the plunger and the end portion of the shaft, g) a slider located on a rail on the interior of the housing, being adapted to move along the rail in a first direction to compress the spring 228 and wherein decompressing of the spring 228 causes the plunger and the lancet holder 233 to rapidly move in a second direction opposite the first direction, and h) a lancet button or operation handle 218 for the lancet to be released and pierce the finger of a user. A test strip 222 being adapted to assist in the determination of an analyte concentration in the blood sample obtained from the pierced finger of a user, by processing electrochemical signals from the test strip 222 by a printed circuit board (PCB) 224 disposed in the housing and adjacent to said test strip 222 and battery strap 226 of the device.

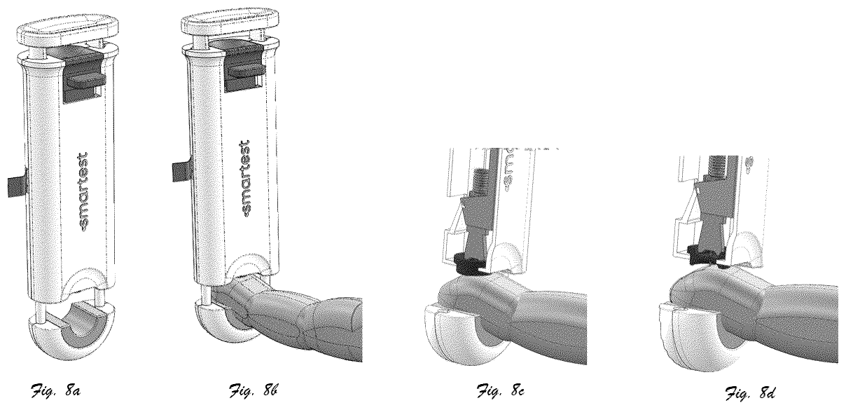

FIG. 8 illustrates a simplified illustration of the steps (a-d) in analyzing a fluid sample by using an integrated diagnostic device, according to a specific embodiment of the present invention. The work flow begins when a user presents a finger with the finger pad facing upwards, wherein the device is in stationary position with the adjustable jaw of the clamping mechanism closed. The user places a thumb on the top of the linear actuator, pressing down, upon which the adjustable jaw opens, and a gap is created between the immobilizer and finger padding (FIG. 8a). While still pressing the thumb down on the linear actuator and maintaining the gap open, the user may place a finger from the second hand on the finger padding, so that the finger pad is facing up. Once positioned correctly, the user may release the thumb pressing the linear actuator, thereby moving the adjustable jaw downwards and closing the gap so that the immobilizer clamps down on the finger pad (FIG. 8b). The finger to be sampled is firmly fixed between the immobilizer and the finger padding, when the user may press a lancet button for the lancet to be released, wherein the retractable lancet pierces the skin of the finger pad clamped between the immobilizer and finger padding (FIG. 8c). The user then again places a thumb on the top of the linear actuator, pressing down, upon which the adjustable jaw opens, and the user may remove the pierced finger from the gap between the immobilizer and finger padding (FIG. 8d), after which the user may release the thumb pressing down the linear actuator and closing the adjustable jaw. Lastly, the user may rotate the finger to an electrochemical sensor opening on upper portion of the device housing. The finger is placed in a sensor cavity to apply a blood drop from the fingertip to a test strip, wherein the test strip being adapted to assist in the determination of an analyte concentration in the blood sample by processing electrochemical signals from the test strip by a printed circuit board (PCB) disposed in the housing and adjacent to said test strip.

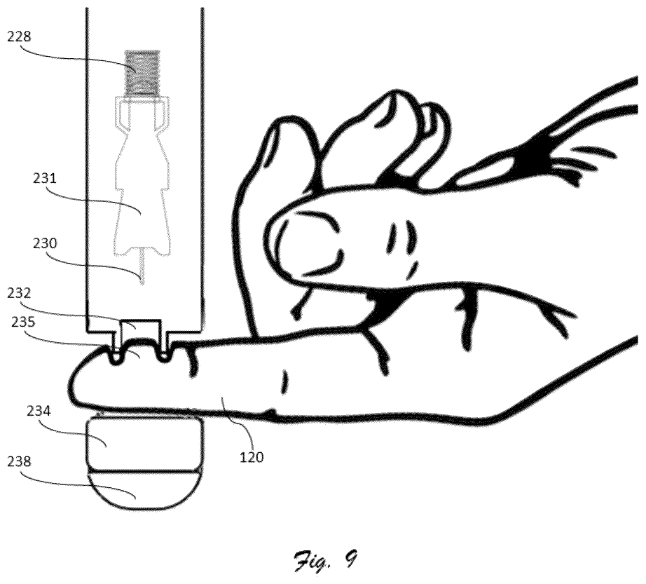

FIG. 9 is a side perspective view of a finger to be pierced with an integrated diagnostic device for analyzing a fluid sample, according to another embodiment of the present invention. A user may place a finger 120 facing up against an immobilizer or finger mount 232 on an upper side and finger padding 234 on a lower side. The immobilizer or finger mount 232 may also serve as a lancet needle guide. The finger padding is comprised of a front and rear 238 finger pad holder. Once the finger 120 is positioned correctly, the user may release the thumb pressing the linear actuator, thereby moving the adjustable jaw of the clamping mechanism downwards and closing the gap so that the immobilizer or finger mount 232 clamps down on the finger padding 234. The finger 120 to be sampled is firmly fixed between the immobilizer 232 and the finger padding 234, creating a “bubble” 235 in the finger pad, increasing local blood flow while maintaining a thinner skin surface at the puncture site. This hemodynamic effect helps to reduce the pain experienced during the lancing, as a well-prepared site allows for a more effective and less traumatic penetration. Then the user may activate the lancing mechanism comprising a spring 228 at least partially surrounding the lancet shaft, and a lancet mesh 231 configured to store the lancet 230. A lancet button may be pressed for the lancet to be released, wherein the retractable lancet pierces the skin of the finger pad clamped between the immobilizer or finger mount 232 and the finger padding 234.

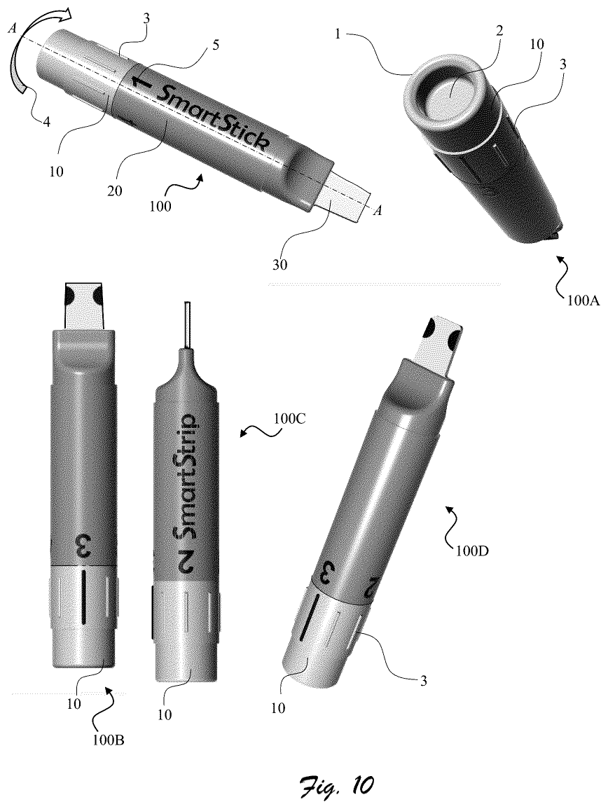

Reference is now made to FIG. 10, schematically illustrating in both a non-limiting and in out of scale manners a medical device (100, 100A-100D) useful for extracting body fluid for further analyses in order to make a diagnosis or to monitor the concentration of analytes, according to one embodiment of the invention. In one embodiment of the invention, the device is a, or a part of a system for testing analyte levels, including glucose, comprising a chip configured for both (i) to process electrical pulses obtained from a communicable analyzing strip, and (ii) to communicate the processed digital data.

As an example, the system is provided as a-or as a part of a device, utilizable as a glucometer, for testing (capillary) blood glucose concentration: it is a small and portable, ready-to-use, single-use, disposable glucometer characterized by both non-tedious and intuitively-operated, free of pain and anxiety for pain or discomfort, and improved user experience.

Glucometer 100 in an elongated device having a main longitudinal axis A:A, in one embodiment of the invention, lateral cross-section (perpendicular to axis A:A) of the device is circular or oval. Alternatively, at least a portion of the cross section is polygonal (not shown). The glucometer comprises, inter alia, a head portion (10), a body portion (20) and analyzing strip (e.g., diabetes test strip30). Various diabetes test strips are commercially available, including e.g., Accu-Chek Aviva Plus™ test strips or Accu-Chek SmartView™ test strips, by Roche Diabetes Care, Inc. (US); FreeStyle™ Abbott Precision Xtra™ Strips, by Abbott (US); GE200™ Test Strips by GE (US).

The head portion may comprise gripping protrusions (3) enabling rotation (4) of the head portion around the main axis relatively to the body. Rotation of the head may facilitate or otherwise enable actuation of a lancet (not shown, positioned, e.g., fleshy underside of the end of a body portion to be pierced, e.g., a finger pad). The lancet is accommodated within portions of the head and/or body, and positioned in parallel with the main axis, e.g., in a concentric or eccentric manner. As the head is potentially rotatable, indication means (5) are provided. illustration 100A shows the same. Head portion 10 comprises a finger pad having an inner embedded portion (2), configured to accept the activated lancet (not shown), and a protruded circumference (1).

It is well in the scope of eth invention where medical device, system. Module or kit 100, also denoted as SmartStrip™, Smart-Lancet™, and/or SmarTest™, may be useful for one or more of the following: (i) collecting analytes and biological samples, (ii) measuring the same so that it is characterized quantitatively vs quantitively to define a value(s) or range thereof, concentration(s) or range thereof; and further (iii) processing/translating by a processor and/or communicating the result(s) to a database, ROM etc., so that the current condition (e.g., in light of a baseline) of the user is obtained. In case of a glucometer, device 100 samples, tests, presents user's current glucose level (condition) in light of glucose boundaries.

It is in the scope of one set of embodiments of the invention wherein at least one portion of circumference 1 is finger-pad is one or more of the following: elastic, configured to deform, narrow or otherwise retract along the axis. Additionally, or alternatively, the elasticity of the finger-pad is characterized by a Young's modulus ranging from any of the following: about 10³ to about 5*10⁹ Pa; from about 10³ to about 10⁸ Pa; from about 10² to about 10⁹ Pa; or from about 10² to about 10⁸ Pa.

It is in the scope of the invention wherein the elastic finger-pad is made of, or otherwise comprises polymers having Young modulus (MPa) and elongation (%) such as polyethylene LDPE 172-282, 100-650; polypropylene 1138-1551, 100-600; polyurethane 0.17-34.47, 250-800; polyethylene terephthalate 2578-4137, 30-300; polyamide (PA66) 1586-3447, 150-300; rubbers, silicone, and any mixture thereof.

The Shore 00 Hardness Scale measures rubbers and gels that are very soft. The Shore A Hardness Scale measures the hardness of flexible mold rubbers that range in hardness from very soft and flexible, to medium and somewhat flexible etc. Hence, additionally, or alternatively to the defined above, the elasticity of the finger-pad is characterized by Shore A ranging from any of the following: 0 to about 30; from 0 to about 40; from 0 to about 50; from about 10 to about 30 or from about 10 to about 45 Shore A; Additionally, or alternatively to the defined above, the elasticity of the finger-pad is characterized by Shore 00 ranging from any of the following: from 0 to about 80; from about 10 to about 70; from about 20 to 6about 0; or from about 20 to about 80 Shore 00.

The finger-pad ensures, by means of its size, shape, elasticity and materials, that the skin to be pierced is provided in a tension so that it is held firmly and the skin is thin as possible. This method of providing the skin to be pierced in tension reduces both pain and fear-of-pain.

It is still in the scope of this set of embodiments wherein the finger-pad is having an outer movable protruding circumference shaped by means of size and shape to snugly fit the body portion to be pierced, e.g., a finger pad, and an inner hollow recess.

Additionally, or alternatively, the finger-pad comprises a static portion and an outer dynamically active protruding circumference, interconnected by means selected from a group consisting e.g., a spring, a sliding mechanism, a rack and pinion mechanism, a screw mechanism. a pneumatic mechanism, a motor, and any combination thereof.

It is also in the scope of the invention wherein finger-pad according to one set of embodiments, is configured by e.g., its size, shape, texture, composition, elasticity, movability and mechanism of action, to reduce skin elasticity before piercing. It is also in the scope of the invention wherein the finger-pad, according to yet another set of embodiments, allows the lancet to pierce the skin in low momentum, and/or low impulse (J, integral of a force over time).

It is further in the scope of the invention wherein finger-pad according to one set of embodiments, is configured by e.g., its size, shape, texture, composition, elasticity, movability and mechanism of action, to press the skin and thus to increase sensation in the piercing region before, whilst and somewhat after lancet pierces the skin.

It is further in the scope of the invention wherein finger-pad according to one set of embodiments, is configured by e.g., its size, shape, texture, composition, elasticity, movability and mechanism of action, to press the skin and thus to increase local blood (capillary blood-) pressure in the piercing region before and whilst lancet.

It is further in the scope of the invention wherein finger-pad according to one set of embodiments, is configured by e.g., its size, shape, texture, composition, elasticity, movability and mechanism of action, to one or more member of a group consisting of (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin.

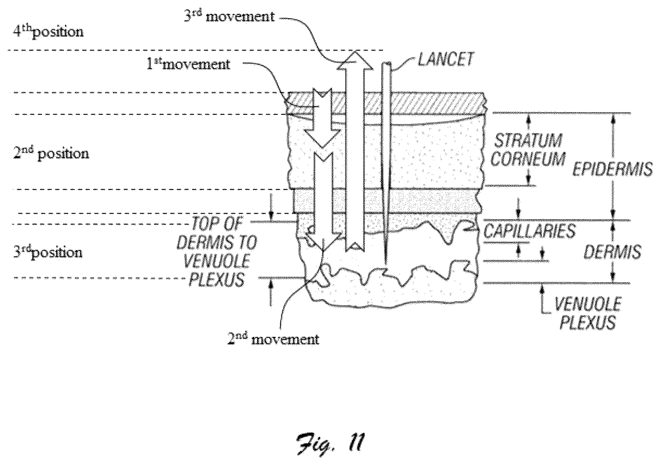

Reference is now made to FIG. 11, adapted from U.S. Pat. No. 9,226,699B2. FIG. 11 schematically illustrating cross-section of a body portion to be pierced. Lancet is protruding device's bottom portion 1 and is penetrating the epidermis (stratumcorneum) and portion of the dermis towards the blood capillaries, yet not getting deep enough to reach the venuole plexus.

It is an object of the invention to lower skin elasticity, use a low momentum piercing and hence to reduce unnecessary pain and lower existing piercing pain; to increase effectiveness of a singly piercing of the finger thus to avoid further piercing of the skin. Hence it is well within the scope of a set of embodiments wherein a method for piercing the body is provided with various steps: e.g., a step of providing a body piercing device with a main longitudinal axis A:A; further providing said device a lancet reversibly movable in parallel with said axis from a first retract position, to a fully extended third position within the dermis and above and outside the venuole plexus, via a second position, located within the stratum cornea, and then back, to a final retracted (safely locked-) fourth position. The positions and movements are schematically presented in the left side FIG. 11.

It is in the scope of another set of embodiments of the invention, wherein the velocity of said lancet in its way from said first to said second position (V_{1st-2nd}) is relatively slow, namely of the following: V_{1st-2nd}≥ about 2 m/s; V_{1st-2nd}≥ about 3 m/s; V_{1st-2nd}≥ about 1 m/s or V_{1st-2nd}≥ about 0.5 m/s. Additionally, or alternatively, the acceleration of the lancet in its way from said first to the second position (A_{1st-2nd}) is relatively low, namely one of the following: A_{1st-2nd}≥ about 4 m/s²; A_{1st-2nd}≥ about 5 m/s²; A_{1st-2nd}≥ about 3 m/s²; A_{1st-2nd}≥ about 2 m/s²or A_{1st-2nd}≥ about 1 m/s².

In FIG. 11, it is in the scope of another set of embodiments wherein one of the following is held true regarding the function of the width of the epidermis W_{EPI}, so that: about 0.25≤ϕ*W_{EPI}≥ about 0.95; about 0.15≤ϕ*W_{EPI}≥ about 0.75; about 0.05≤ϕ*W_{EPI}≥ about 0.75 or about 0.15≤ϕ*W_{EPI}≥ about 0.85. Additionally, or alternatively, one of the following is held true regarding the function of the width of the stratum corneum W_{SC}, so that: about 0.3≤ϕ*W_{EPI}≥ about 0.6, about 0.3≤ϕ*W_{EPI}≥ about 0.7; about 0.3≤ϕ*W_{EPI}≥ about 0.8; about 0.2≤ϕ*W_{EPI}≥ about 0.6; about 0.1≤ϕ*W_{EPI}> about 0.6; or about 0.1<ϕ*W_{EPI}≥ about 0.75.

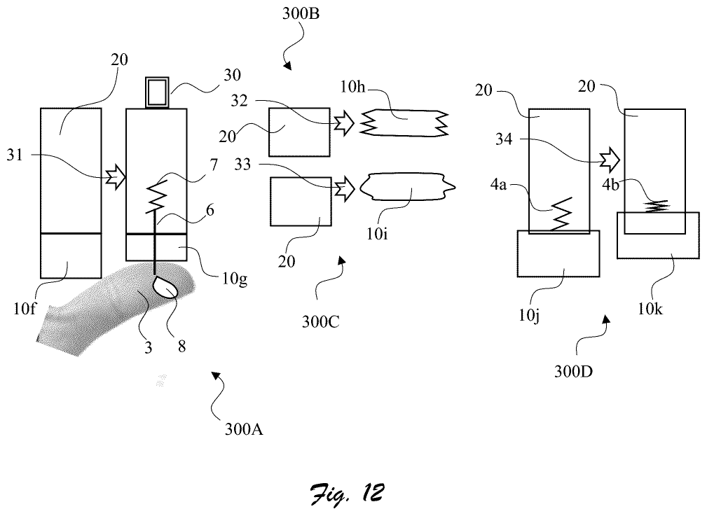

Reference is now made to FIG. 12, schematically illustrating an actuation mechanism of head portion (10, see 10f-10k). Image 300A depicts an out-of-scale presentation of static body portion 20 and movable head portion, (i) before onset (10f); and (ii), at time thumb presses elastic/movable finger-pad 10g; and lancet 6, activated by a spring mechanism 7, is piercing body (3). At its 3^{rd} position, lancet penetrates the capillaries, and ejecting minimal volume of blood (8) to be detected e.g., by means of analyzing strip 30. Image 300B depicts an out-of-scale presentation of an accordion-like reversibly foldable finger-pad 10h. Image 300C similarly depicts an out-of-scale presentation of either reversible or irreversible Poisson deformation of an clastic bend 10i. Image 300D depicts an out-of-scale presentation of finger-pad, comprises a static portion and an outer dynamically active protruding circumference, interconnected by a spring: 4a before thumb pressing the head portion 10j; and 4b at time thumb presses head portion 10k.

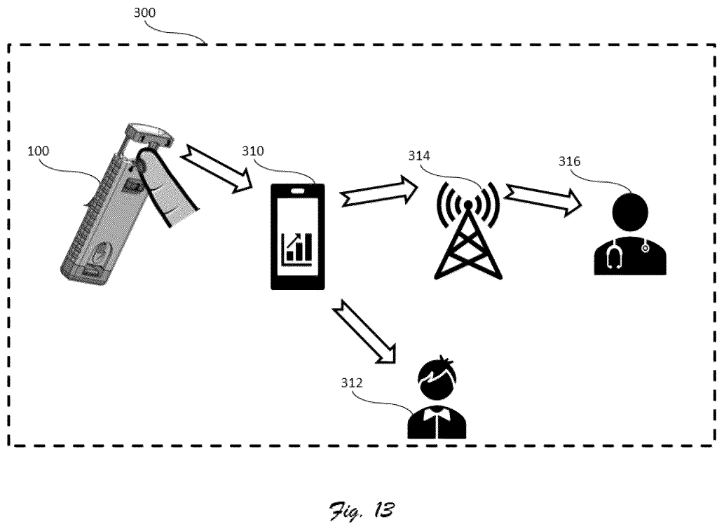

Reference is now made to FIG. 13, schematically illustrating a communicable system 300, comprising one, two or many users 312, each of which is either patient or healthy person. User test himself/herself periodically by device 100.

Test strip is imaged or otherwise identified and read by a detector ad processor e.g., a smartphone of any other communicable IoT. In one of the embodiments, the specification of the readout 310 is further processed, presented to the user 312 and affiliates thereof, and/or communicated to a remote location 314, e.g., to medical insurer, authorized person, or medical center 316, following one or a plurality of steps of dentification, authorification, authentication, and verification.

Following the hereto disclosed example of glucometer 100, by periodically testing so-called healthy people (and NOT only patients), this new type of painless (or reduced anxiety-) glucometer suit best people with needle fear, hence encourages most of the population, namely those who have had none previous indications for the disease, to test their sugar levels periodically and hence for enabling early detection of diabetes.

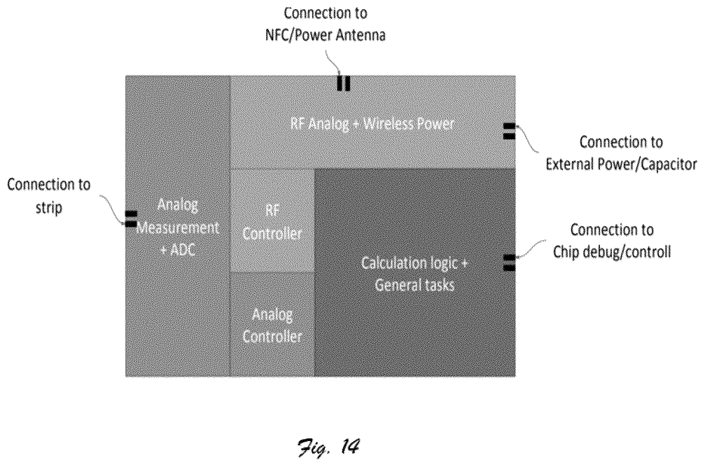

A schematic illustration of a chip (e.g., between 20 to 200 nm) according to yet another embodiment of the invention is depicted in FIG. 14. Scheme of the chip defines (i) its connectability with either or both (a) antenna for communicating the processed digital data to user and to remote locations, and (b) energy source; (ii) to a communication cable; (iii) access to revising, updating and debugging data and software imbedded within the chip.

The chip is configured to be charged either by wireless means e.g., a cellular telephone device, or in a cordial manner. the chip is communicable with electrochemical strip so that a digital readout of sugar level is obtained to the user, e.g., by a mobile application, to a remote-location, e.g., to insurers (including elevated test's cost), medical practitioners etc. The hereto disclosed glucometer solves the uneasiness of commercially available products for both user and is/her insurer; and significantly reduces user's pain, time lost, need for multi-piercing for achieving minimal volume of blood, tedious bureaucracy, comfortable for everyday Keep & Carry etc. The hereto disclosed unified (all-in-one housing) blood level testing system further ensures both relatively low production costs and reduced use costs as the processor (the chip) is not expensive and hence its low price enables the device to be single use, inexpensive for both the user and medical insurer. The hereto disclosed glucometer is a unified well integrated hybrid all-in-one testing system, comprising glucometer, lancet and analyzing strip. This minimal size device is easy to manufacture, storage, distribution and marketing. Its novel lancet system and operation significantly reduce both pain and fear-of-pain.

It is within the scope of the present invention to provide configured needles and methods for repeatedly withdrawing blood from a subject to follow the sugar levels over time such as 0.5 min, 10, 15, 20 mins and so on in order to estimate the patient's state of sugar metabolism.

It is within the scope of the present invention to provide needles configured for extracting and measuring blood sucrose, glucose and fructose.

It is within the scope of the present invention to provide devices and software for deriving positive synergic values concerning health using real time inputs of sugar levels, cholesterol levels, blood pressure levels and glaucoma measurements by measuring and controlling at least one of these values the patient's health may be improved.

It is within the scope of the present invention to provide needle devices, software and methods for estimating real time and ongoing subject tolerance or vulnerability to various sugar rich foods such as ice cream and corn products.

It is within the scope of the present invention to provide secure cloud or local internet communications network and data storage and processing between health insurer, care providers, physicians and hospitals, optionally also with patient, comprising an insurance premium calculation module based on transformation of collected data to actuarial values.

It is within the scope of the present invention to provide a biometric fingerprint verification module comprising data collected and processed by the method of the present invention.

It is within the scope of the present invention to provide devices, methods and software for deriving predictions of long-term patient quality of life and potential states of hypoglycemia, hyperglycemia, ketoacidosis, acute diabetes and other related conditions.

It is within the scope of the present invention to provide a needle device wherein the piercing part of the needle and a surrounding ring or cuff are configured to come into contact with the skin. The ring or cuff which surrounds the needle is configured to vibrate or otherwise distractedly stimulate the skin surface during the needle piercing procedure in order to reduce or mitigate perceived needle pain by units indicated on, for example, the Wong Baker Pain Scale.

It is within the scope of the present invention to provide the needle devices and methods to predict or alert subjects engaged in physical sports by taking and processing analytes before, during or after an individual engages in such sport, processing collected data and informing the subject or caregiver or sports authority or supervisory authority.

### Case Report Illustrating Aspects of the Present Invention.

This is a case report of a young healthy pregnant woman, on her first pregnancy, that illustrates the need for the aspect of the present invention concerned with in checking for diabetes during pregnancy.

At the beginning of her pregnancy, at about her 12^{th} week of gestation, she was found to have normal fasting glucose level of 80 mg %. There was no history of diabetes in her close family. The next glucose test, glucose challenge test (GCT), that was taken on her 24^{th} week of gestation, showed abnormal value of 300 mg %. She was then sent to the High Risk Pregnancy clinic, but within few days, before reaching the clinic, she was admitted to the emergency room with severe ketoacidosis. Going forward she was delivered by emergency cesarean section. The new born was severely handicapped.

The aforementioned case shows that more frequent blood glucose tests as provide by the present invention could have helped in detecting the situation at an earlier stage, avoiding the severe outcome. Considering the difficulty and expenses of being admitted every week to the clinic to check for blood glucose level or having every pregnant woman purchasing and carrying a personal glucose meter, it seems that the idea of a single use self-testing, painless and low-cost blood glucose test, SmarTest, would solve these problems.

While this invention has been described as having a preferred design, the present invention can be further modified within the spirit and scope of this disclosure. The application is therefore intended to cover any variations, uses, or adaptations of the invention using its general principles. Further, this application is intended to cover such departures from the present disclosure that arise from known or customary practice and the art to which this invention pertains and which fall within the limits of the appended claims.

1. An integrated diagnostic device for reducing or eliminating pain during body fluid sampling of a patient, comprising:
a. an enclosure forming a housing with inner walls, outer walls and a plurality of sub-units within said enclosure; said sub-units configured to complete an operation related to said body fluid sampling;
b. at least one pain reduction mechanism for alleviating pain associated with the penetrating of the skin by a penetrating member;
c. a skin-penetrating mechanism comprising a penetrating member accommodated within said housing and configured to penetrate a skin of said patient.

2. The integrated diagnostic device of claim 1, wherein said at least one pain reduction mechanism is a clamping mechanism, comprising:
a. a linear actuator extending from an upper or lower portion of said housing and moved by a patient finger; said linear actuator includes springs configured to facilitate linear motion, and open an adjustable jaw 110 in an upper or lower portion of said housing, and ensure uniform clamping pressure;
b. an adjustable jaw 110 configured to move along a linear path and apply a clamping force on a finger 120 upon release of said linear actuator; and
c. an immobilizer 122 on an upper side and finger padding 124 on a lower side of said adjustable jaw configured to secure said finger in place, so that an upper cushion of said finger 120 is aligned with said skin-penetrating mechanism.

3. The integrated diagnostic device of claim 2, wherein said housing 111 comprising:
a. an electrochemical sensor opening 118 formed therein;
b. an electrochemical sensor pack having a sensor cavity, said sensor cavity being adapted to house a communicable test sensor 114 therein;
c. a communicable test sensor 114 being adapted to assist in the determination of an analyte concentration in the fluid sample;
d. a printed circuit board (PCB) 16 disposed in said housing 111 and adjacent to said communicable test sensor 114;
e. one or more members of a group consisting of power source, power-source adapter, power source input means; means for input communication, means for output communication and any combination thereof; and
f. optionally having an exterior with a grip 113.
wherein said device is configured to (i) process electrical pulses obtained from said communicable test sensor, and (ii) communicate the processed digital data.

4. The integrated diagnostic device of claim 3, wherein said skin penetrating mechanism comprising:
a. a lancet 112 moveable between a standby position, an extended position, and a testing position;
b. a lancet mesh 126 configured to store said lancet 112;
c. a lancet holder 128 adapted to removably engage a base of said lancet 112,
d. a plunger coupled to said lance holder 128, said plunger having a central portion;
e. a shaft running through said central portion of the plunger, said plunger being adapted to move along said shaft, said shaft having an end portion that is adapted to secure said shaft to said integrated diagnostic device;
f. a spring 130 at least partially surrounding said shaft, said spring 130 being located between said plunger and said end portion of the shaft; and
g. a slider located on a rail on the interior of said housing 111, said slider being adapted to move along said rail in a first direction to compress said spring 130 and wherein decompressing of said spring 130 causes said plunger and said lance holder 128 to rapidly move in a second direction opposite the first direction; and
h. a lancet button 132 for the lancet to be released.

5. The integrated diagnostic device of claim 1, wherein said at least one pain reduction mechanism is an ergonomically contoured elastic finger-pad; said ergonomically contoured elastic finger-pad is a concaved member, having an outer elastic protruding circumference shaped by means of size and shape to snugly fit said finger pad, and an inner hollow recess configured to accept the sharp end of said penetrating member.

6. The integrated diagnostic device of claim 5, wherein size, shape, elasticity and materials of said ergonomically contoured elastic finger-pad is further configured to:
a. ensure, to either or both, (i) that body portion to be penetrated is provided in a tension so that it is held firmly and the skin is thin as possible, and (ii) said tension in effective to increase blood pressure in at least 5 mmHg as compared to local blood pressure before tensioning said body portion;
b. provide elasticity of said finger-pad ranging from Young's modulus: 10³ to 5*10⁹Pa; from 0 to 45 Shore A; or 0 to 80 Shore 00;
c. deform when pressed by a body portion to be pierced to a width W_{FPR}, equal or smaller than a function of the width of the epidermis W_{EPI}, so that W_{FPR}≤ϕ*W_{EPI}, 0.25≤ϕ≥0.75; and
d. deform when pressed by a body portion to be pierced to a width W_{FPR}, equal or smaller than a function of the width of the stratum corneum W_{SC}, so that c≤φ*W_{SC}, 0.3≤φ≥0.6.

7. The integrated diagnostic device of claim 6, wherein said ergonomically contoured elastic finger-pad is further configured to one or more members of a group consisting of (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin.

8. The integrated diagnostic device of claim 7, wherein said skin penetrating mechanism comprising a lancet reversibly movable in parallel with a main longitudinal axis A:A of said device from a first retract position, to a fully extended third position within the dermis and above and outside the venuole plexus, via a second position, located within the stratum cornea, and then back, to a final retracted (safely locked-) fourth position; wherein the velocity of said lancet in its way from said first to said second position (V_{1st-2nd}) is relatively slow, namely V_{1st-2nd}≥2 m/s.

9. The integrated diagnostic device of claim 8, wherein said housing accommodating:
a. a chip;
b. a lancet;
c. one or more analyte or analytes analyzing strips; and
d. one or more members of a group consisting of power source, power-source adapter, power source input means; means for input communication, means for output communication and any combination thereof.

10. The integrated diagnostic device of claim 1, further comprising a wireless communication interface disposed within said housing 111 and coupled to a controller, wherein said controller is adapted to wirelessly communicate with a remote device to provide a graphical user interface (GUI) for orchestrating a test of the performance metric of said body fluid and displaying results of said analysis.

11. The integrated diagnostic device of claim 10, wherein said housing 111 is a disposable member.

12. A body-piercing device for reducing or eliminating pain during piercing of the body, comprising:
a. an enclosure forming a housing with inner walls, outer walls and a plurality of sub-units within said enclosure; said sub-units configured to complete an operation related to said body fluid sampling;
b. at least one pain reduction mechanism for alleviating pain associated with the penetrating of the skin by a penetrating member;
c. a skin-penetrating mechanism comprising a penetrating member accommodated within said housing and configured to penetrate a skin of said patient.

13. The body-piercing device of claim 12, wherein said at least one pain reduction mechanism is a clamping mechanism, comprising:
a. a linear actuator extending from an upper or lower portion of said housing and moved by a patient finger; said linear actuator includes springs configured to facilitate linear motion, and open an adjustable jaw 110 in an upper or lower portion of said housing, and ensure uniform clamping pressure;
b. an adjustable jaw 110 configured to move along a linear path and apply a clamping force on a finger 120 upon release of said linear actuator; and
c. an immobilizer 122 on an upper side and finger padding 124 on a lower side of said adjustable jaw configured to secure said finger in place, so that an upper cushion of said finger 120 is aligned with said skin-penetrating mechanism.

14. The body-piercing device of claim 13, wherein said skin-penetrating mechanism comprising:
a. a lancet 112 moveable between a standby position, an extended position, and a testing position;
b. a lancet mesh 126 configured to store said lancet 112;
c. a lancet holder 128 adapted to removably engage a base of said lancet 112,
d. a plunger coupled to said lance holder 128, said plunger having a central portion;
e. a shaft running through said central portion of the plunger, said plunger being adapted to move along said shaft, said shaft having an end portion that is adapted to secure said shaft to said integrated diagnostic device;
f. a spring 130 at least partially surrounding said shaft, said spring 130 being located between said plunger and said end portion of the shaft; and
g. a slider located on a rail on the interior of said housing 111, said slider being adapted to move along said rail in a first direction to compress said spring 130 and wherein decompressing of said spring 130 causes said plunger and said lance holder 128 to rapidly move in a second direction opposite the first direction; and
h. a lancet button 132 for the lancet to be released.

15. The body-piercing device of claim 14, further comprising an integrated diagnostic mechanism characterized by:
a. an electrochemical sensor opening 118 formed therein;
b. an electrochemical sensor pack having a sensor cavity, said sensor cavity being adapted to house a communicable test sensor 114 therein;
c. a communicable test sensor 114 being adapted to assist in the determination of an analyte concentration in the fluid sample;
d. a printed circuit board (PCB) 16 disposed in said housing 111 and adjacent to said communicable test sensor 114; and
e. one or more members of a group consisting of power source, power-source adapter, power source input means; means for input communication, means for output communication and any combination thereof;
wherein said body-piercing device is configured to (i) process electrical pulses obtained from said communicable test sensor, and (ii) communicate the processed digital data.

16. The body-piercing device of claim 14, wherein said at least one pain reduction mechanism is an ergonomically contoured elastic finger-pad; said ergonomically contoured elastic finger-pad is a concaved member, having an outer elastic protruding circumference shaped by means of size and shape to snugly fit said finger pad, and an inner hollow recess configured to accept the sharp end of said penetrating member.

17. The body-piercing device of claim 16, wherein size, shape, elasticity and materials of said ergonomically contoured elastic finger-pad is further configured to:
a. ensure, to either or both, (i) that body portion to be penetrated is provided in a tension so that it is held firmly and the skin is thin as possible, and (ii) said tension in effective to increase blood pressure in at least 5 mmHg as compared to local blood pressure before tensioning said body portion;
b. provide elasticity of said finger-pad ranging from Young's modulus: 10³ to 5*10⁹Pa; from 0 to 45 Shore A; or 0 to 80 Shore 00;
c. deform when pressed by a body portion to be pierced to a width W_{FPR}, equal or smaller than a function of the width of the epidermis W_{EPI}, SO that W_{FPR}≤ϕ*W_{EPI}, 0.25≤ϕ≥0.75; and
d. deform when pressed by a body portion to be pierced to a width W_{FPR}, equal or smaller than a function of the width of the stratum corneum W_{SC}, so that c≤φ*W_{SC}, 0.3≤φ≥0.6.

18. The body-piercing device of claim 17, wherein said ergonomically contoured elastic finger-pad is further configured to one or more members of a group consisting of (i) pressing skin region to be pierced, hence increasing sensation in the piercing region before, whilst and somewhat after lancet pierces the skin; (ii) pressing skin region to be pierced, hence decreasing skin's elasticity before and whilst lancet pierces the skin; and (iii) pressing the skin region to be pierced, hence increasing local blood (capillary blood-) pressure in the piercing region before and whilst lancet pierces the skin.

19. The body-piercing device of claim 17, wherein said skin penetrating mechanism comprising a lancet reversibly movable in parallel with a main longitudinal axis A:A of said device from a first retract position, to a fully extended third position within the dermis and above and outside the venuole plexus, via a second position, located within the stratum cornea, and then back, to a final retracted (safely locked-) fourth position; wherein the velocity of said lancet in its way from said first to said second position (V_{1st-2nd}) is relatively slow, namely V_{1st-2nd}≥2 m/s.

20. A method for reducing or eliminating pain during body fluid sampling of a patient, comprising steps of:
a. obtaining the integrated diagnostic device of claim 1;
b. actuating an adjustable jaw 110 of a finger clamp along a linear path to open a fingertip aperture of said housing 111;
c. placing a finger 120 on the lower side of said fingertip aperture on finger padding 124;
d. releasing said adjustable jaw 110 to close said fingertip aperture and apply clamping pressure on said finger 120;
e. pressing a lancet button 132 to release a lancet 112 configured to pierce said finger 20 through said finger clamp and to allow blood of said pricked finger accumulate on said fingertip;
f. actuating said adjustable jaw 110 of said finger clamp to open said fingertip aperture;
g. retrieving said finger 120 from said fingertip aperture;
h. rotating said finger 120 to an electrochemical sensor opening of said housing 111;
i. placing said finger 120 in a sensor cavity to apply a blood drop from said fingertip to a test strip 114 therein; said test strip 114 being adapted to assist in the determination of an analyte concentration in said fluid sample;
j. processing electrochemical signals from said test strip 114 by a printed circuit board (PCB) 116 disposed in said housing 111 and adjacent to said test strip 114; and
k. communicating processed digital data.
